(19) United States
(12) Patent Application Publication
(10) Pub. No.: US 2026/0159826 A1
(43) Pub. Date: Jun. 11, 2026

(54) METHOD OF MODIFYING CELLS

(71) Applicant: ROYAL MELBOURNE INSTITUTE OF TECHNOLOGY, Melbourne (AU)

(72) Inventors: Leslie YEO, Melbourne (AU); Lizebona August AMBATTU, Melbourne (AU)

(21) Appl. No.: 19/129,230

(22) PCT Filed: Nov. 17, 2023
(86) PCT No.: PCT/AU2023/051172

§ 371 Date: May 12, 2025

(30) Foreign Application Priority Data
Nov. 18, 2022 (AU) 2022903491

(51) Int. Cl. C12N 13/00; A61K 35/52; A61K 35/54; A61K 40/11; A61K 40/17; A61K 40/31; C12N 5/0786

(52) U.S. Cl. CPC C12N 13/00; A61K 35/52; A61K 35/54; A61K 40/11; A61K 40/17; A61K 40/31; C12N 5/0645; C12N 2506/115

(57) ABSTRACT

Provided are methods and associated apparatus for increasing calcium mobilisation in a cell. Calcium mobilisation is through exposing the cells to acoustic wave energy such as through surface acoustic waves (SAW) and surface reflected bulk waves (SRBW). Included are methods of treating immune cells to activate the immune cells, increase cytokine production and differentiate monocytes to macrophages. Also included are methods involving exposing ova and/or sperm to acoustic wave energy for in vitro fertilisation and/or artificial insemination.

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

The present application claims priority from AU 2022903491 filed 18 Nov. 2022, the entire contents of which are incorporated herein by reference.

### FIELD OF THE DISCLOSURE

Embodiments generally relate to methods and associated apparatus for increasing calcium mobilisation in a cell. Also provided are a method of increasing an immune response in a subject, a method of in vitro fertilisation and a method of artificial insemination.

### BACKGROUND

Calcium (Ca2+) signalling is an important function that is responsible for communicating and driving a variety of intracellular processes. When released from its internal stores, calcium exerts allosteric regulatory effects on many enzymes and proteins. Calcium can act in signal transduction resulting from activation of ion channels or as a second messenger caused by indirect signal transduction pathways. This calcium mobilisation also triggers the influx of calcium from the external medium into the cell as a means of further propagating calcium signals and also replenishing depleted pools of calcium.

Infertility is currently one of the most frequent health concerns facing the population aged 25-45 years. Assisted reproductive technologies, such as in vitro fertilisation and artificial insemination, are procedures that involve extracorporeal handling of both human eggs (oocytes or ova) and sperm (spermatozoa), and of embryos for the purpose of establishing a pregnancy in a female subject. Such assisted reproductive technologies are also widely used for veterinary purposes and in livestock industries. However, successful pregnancy rates obtained with assisted reproductive technology techniques remain relatively low.

Immune cell therapy, such as CAR T-cell therapy, is a form of immunotherapy that uses altered immune cells (e.g. T-cells) to target and kill cancer cells. Although these therapies are promising, efficacy and potency challenges remain.

There is a need for methods of modifying cells to provide improved cellular function.

### SUMMARY OF THE DISCLOSURE

The present inventors have identified that exposing cells to acoustic wave energy increases calcium mobilisation. Further, the increased calcium mobilisation improves the function of distinct cells types, for example improved fertilisation and improved immune responses.

In an example, the disclosure provides a method of increasing calcium mobilisation in a cell comprising exposing the cell to acoustic wave energy. In an example, the disclosure provides a method of increasing calcium mobilisation in a mammalian cell comprising exposing the cell to acoustic wave energy.

In an example, cell is selected from an immune cell, an ovum or sperm.

In an example, the disclosure provides a method of fertilising an ovum, the method comprising 1) exposing an ovum to acoustic wave energy, and 2) fertilising the ovum with a sperm, and/or 3) exposing sperm to acoustic wave energy, and 4) fertilising an ovum with the sperm.

In an example, the disclosure provides a method of fertilising an ovum, the method comprising
1) exposing the ovum to acoustic wave energy, and
2) fertilising the ovum with a sperm.

In an example, the disclosure provides a method of fertilising an ovum, the method comprising
1) exposing sperm to acoustic wave energy, and
2) fertilising the ovum with the sperm.

In an example, the disclosure provides a method of fertilising an ovum, the method comprising exposing the ovum to acoustic wave energy, and fertilising the ovum with a sperm. In an example, the disclosure provides a method of fertilising an ovum, the method comprising exposing sperm to acoustic wave energy, and fertilising the ovum with the sperm. In an example, the disclosure provides a method of fertilising an ovum, the method comprising exposing the ovum and the sperm to acoustic wave energy, and fertilising the ovum with the sperm.

The present disclosure also contemplates that exposing an oocyte to acoustic wave energy triggers maturation into ovum. Accordingly, in the above examples, the method further comprises the step of exposing an oocyte to acoustic wave energy according to the methods disclosed herein.

In an example, the disclosure provides a method of activating an immune cell, the method comprising exposing the cell to acoustic wave energy. In an example, the disclosure provides a method of activating an immune cell, the method comprising exposing the immune cell to acoustic wave energy.

In an example, the disclosure provides a method of increasing cytokine production of an immune cell, the method comprising exposing the cell to acoustic wave energy. In an example, the disclosure provides a method of increasing cytokine production of an immune cell, the method comprising activating the immune cell by exposing the immune cell to acoustic wave energy. In an example, the cytokine is one or more of IL-2, IL-6, and/or tumour necrosis factor (TNF)-alpha.

In an example, the immune cell is a T cell, B cell, dendritic cell, a monocyte, or a natural killer cell. In an example, the immune cell comprises an exogenous chimeric antigen receptor (CAR). In an example, the immune cell is a CAR-T cell. In an example, the immune cell is a CAR-M cell. In an example, the immune cell is a B cell. In an example, the immune cell is a monocyte. In an example, the activation of the monocyte is the differentiation of the monocyte into a macrophage.

In an example, the acoustic wave energy is provided in the form of a bulk acoustic wave (BAW).

In an example, the acoustic wave energy is provided in the form of surface acoustic waves (SAW).

In an example, the acoustic wave energy is provided in the form of surface reflected bulk waves (SRBW).

In an example, the cell, or a population thereof, are in a cell culture medium. In an example, the cell or a population thereof, the ovum or a population thereof, the sperm or a population thereof, or the immune cell or a population thereof, are in a cell culture medium. In an example, the ovum or a population thereof are in a cell culture medium.

In an example, the sperm or a population thereof are in a cell culture medium. In an example, the immune cell, or a population thereof are in a cell culture medium.

In an example, the cell, or a population thereof, are in a tissue. In an example, the tissue is in vivo. In an example, the tissue is ex vivo. In an example, the tissue is from a mammalian subject. In an example, the tissue is an ovary. In an example, the tissue is a teste.

In an example, the acoustic wave energy is provided to the cell, or a population thereof, via an apparatus, the apparatus comprising: (i) an acoustic wave generator configured to generate acoustic energy at a selected power and frequency; and (ii) a receptacle for accommodating a cell, or a population thereof, in a culture medium, the receptacle being configured to receive acoustic energy generated by the acoustic wave generator. In an example, the acoustic wave energy is provided to the cell or a population thereof, the ovum or a population thereof, the sperm or a population thereof, or the immune cell or a population thereof, via an apparatus, the apparatus comprising: (i) an acoustic wave generator configured to generate acoustic energy at a selected power and frequency; and (ii) a receptacle for accommodating a cell, or a population thereof, in a culture medium, the receptacle being configured to receive acoustic energy generated by the acoustic wave generator. In an example, the acoustic wave energy is provided to the ovum or a population thereof, via an apparatus, the apparatus comprising: (i) an acoustic wave generator configured to generate acoustic energy at a selected power and frequency; and (ii) a receptacle for accommodating a cell, or a population thereof, in a culture medium, the receptacle being configured to receive acoustic energy generated by the acoustic wave generator. In an example, the acoustic wave energy is provided to the sperm or a population thereof, via an apparatus, the apparatus comprising: (i) an acoustic wave generator configured to generate acoustic energy at a selected power and frequency; and (ii) a receptacle for accommodating a cell, or a population thereof, in a culture medium, the receptacle being configured to receive acoustic energy generated by the acoustic wave generator. In an example, the acoustic wave energy is provided to the immune cell or a population thereof, via an apparatus, the apparatus comprising: (i) an acoustic wave generator configured to generate acoustic energy at a selected power and frequency; and (ii) a receptacle for accommodating a cell, or a population thereof, in a culture medium, the receptacle being configured to receive acoustic energy generated by the acoustic wave generator.

In another example, the acoustic wave generator is in direct contact with the receptacle. In another example, the acoustic wave generator is in direct contact with a tissue. In an example, the tissue is in vivo. In an example, the tissue is ex vivo. In an example, the tissue is from a mammalian subject. In an example, the tissue is from a human subject. In an example, the tissue is an ovary. In an example, the tissue is a teste. In an example, the acoustic wave energy is provided to the cell, or a population thereof, via an apparatus, the apparatus comprising:
(i) an acoustic wave generator configured to generate acoustic energy at a selected power and frequency; and
(ii) a receptacle for accommodating a cell, or a population thereof, in a tissue, the receptacle being configured to receive acoustic energy generated by the acoustic wave generator.

In an example, the receptacle defines a reservoir configured to accommodate the cell, or a population thereof, in a tissue. In an example, at least part of the acoustic wave generator is arranged in direct contact with the tissue. In these examples, the frequency of the applied acoustic energy is in the range of about 1 MHz to about 1 GHz. In an example, the frequency of the applied acoustic energy is between about 1 MHz to about 15 MHz. In an example, the frequency of the applied acoustic energy is between about 1 MHz to about 5 MHz. In an example, the frequency of the applied acoustic energy is about 10 MHz.

In an example, the receptacle defines a reservoir configured to accommodate the cell, or a population thereof, in a culture medium. In an example, the receptacle defines a reservoir configured to accommodate the cell or a population thereof, the ovum or a population thereof, the sperm or a population thereof, or the immune cell or a population thereof, in a culture medium. In an example, the receptacle defines a reservoir configured to accommodate the ovum or a population thereof in a culture medium. In an example, the receptacle defines a reservoir configured to accommodate the sperm or a population thereof in a culture medium. In an example, the receptacle defines a reservoir configured to accommodate the immune cell or a population thereof in a culture medium.

In an example, the acoustic wave generator comprises a piezoelectric substrate defining a working surface and an interdigitated transducer located on and in contact with the working surface of the piezoelectric substrate.

In an example, the acoustic wave energy is propagated as a surface acoustic wave (SAW) along the working surface.

In an example, the acoustic wave energy is propagated as a surface reflected bulk wave (SRBW) within the piezoelectric substrate and internally reflected between the working surface and an adjacent surface of the piezoelectric substrate.

In an example, at least part of the acoustic wave generator is arranged in direct contact with the culture medium. In an example, at least part of the acoustic wave generator is arranged in direct contact with the receptacle. In an example, at least part of the acoustic wave generator is arranged in direct contact with the tissue.

In an example, the acoustic wave generator is separated from the culture medium.

In an example, the receptacle is coupled to the acoustic wave generator with a coupling material.

In an example, the frequency of the applied acoustic energy is in the range of about 1 MHz to about 1 GHz. In an example, the frequency of the applied acoustic energy is about 10 MHz.

In an example, the input power for the acoustic wave generator is in the range of about 0.5 W to about 2.5 W. In an example, the input power for the acoustic wave generator is 0.5 W. In an example, the input power for the acoustic wave generator is 1 W. In an example, the input power for the acoustic wave generator is 1.5 W. In an example, the input power for the acoustic wave generator is 2 W. In an example, the input power for the acoustic wave generator is 2.5 W.

In an example, an acoustic pressure applied to the cell, or a population thereof, by the acoustic wave generator is about 0.1 MPa. In an example, an acoustic pressure applied to the cell, or a population thereof, the ovum or a population thereof, the sperm or a population thereof, or the immune cell or a population thereof, by the acoustic wave generator is about 0.1 MPa. In an example, an acoustic pressure applied to the ovum, or a population thereof, by the acoustic wave generator is about 0.1 MPa. In an example, an acoustic pressure applied to the sperm, or a population thereof, by the acoustic wave generator is about 0.1 MPa. In an example, an acoustic pressure applied to the immune cell, or a population thereof, by the acoustic wave generator is about 0.1 MPa.

In an example, the cell, or a population thereof, is exposed to acoustic wave energy for a period between about 30 seconds to about 60 minutes. In an example, the cell or a population thereof, the ovum or a population thereof, the sperm or a population thereof, or the immune cell or a population thereof, is exposed to acoustic wave energy for a period between about 30 seconds to about 60 minutes. In an example, the ovum or a population thereof, is exposed to acoustic wave energy for a period between about 30 seconds to about 60 minutes. In an example, the sperm or a population thereof, is exposed to acoustic wave energy for a period between about 30 seconds to about 60 minutes. In an example, the immune cell or a population thereof, is exposed to acoustic wave energy for a period between about 30 seconds to about 60 minutes.

In an example, the cell, or a population thereof, is exposed to acoustic wave energy for a period between about 30 seconds to about 30 minutes. In an example, the cell or a population thereof, the ovum or a population thereof, the sperm or a population thereof, or the immune cell or a population thereof, is exposed to acoustic wave energy for a period between about 30 seconds to about 30 minutes. In an example, the ovum or a population thereof, is exposed to acoustic wave energy for a period between about 30 seconds to about 30 minutes. In an example, the sperm or a population thereof, is exposed to acoustic wave energy for a period between about 30 seconds to about 30 minutes. In an example, the immune cell or a population thereof, is exposed to acoustic wave energy for a period between about 30 seconds to about 30 minutes.

In an example, the cell, or a population thereof, is exposed to acoustic wave energy for a period of about 5 minutes or about 10 minutes. In an example, the cell or a population thereof, the ovum or a population thereof, the sperm or a population thereof, or the immune cell or a population thereof, is exposed to acoustic wave energy for a period of about 5 minutes or about 10 minutes. In an example, the ovum or a population thereof, is exposed to acoustic wave energy for a period of about 5 minutes or about 10 minutes. In an example, the sperm or a population thereof, is exposed to acoustic wave energy for a period of about 5 minutes or about 10 minutes. In an example, the immune cell or a population thereof, is exposed to acoustic wave energy for a period of about 5 minutes or about 10 minutes.

In an example, the cell, or a population thereof, is exposed to acoustic wave energy for a period of about 5 minutes. In an example, the cell or a population thereof, the ovum or a population thereof, the sperm or a population thereof, or the immune cell or a population thereof, is exposed to acoustic wave energy for a period of about 5 minutes. In an example, the ovum or a population thereof, is exposed to acoustic wave energy for a period of about 5 minutes. In an example, the sperm or a population thereof, is exposed to acoustic wave energy for a period of about 5 minutes. In an example, the immune cell or a population thereof, is exposed to acoustic wave energy for a period of about 5 minutes.

In an example, the cell, or a population thereof, is exposed to acoustic wave energy for a period of about 10 minutes. In an example, the cell or a population thereof, the ovum or a population thereof, the sperm or a population thereof, or the immune cell or a population thereof, is exposed to acoustic wave energy for a period of about 10 minutes. In an example, the ovum or a population thereof, is exposed to acoustic wave energy for a period of about 10 minutes. In an example, the sperm or a population thereof, is exposed to acoustic wave energy for a period of about 10 minutes. In an example, the immune cell or a population thereof, is exposed to acoustic wave energy for a period of about 10 minutes.

In an example, methods according to the disclosure comprise exposing the cell, or a population thereof, to one or more periods of acoustic wave energy followed by incubation in the absence of acoustic stimulation. In an example, methods according to the disclosure comprise exposing the cell or a population thereof, the ovum or a population thereof, the sperm or a population thereof, or the immune cell or a population thereof, to one or more periods of acoustic wave energy followed by incubation in the absence of acoustic stimulation. In an example, methods according to the disclosure comprise exposing the ovum or a population thereof, to one or more periods of acoustic wave energy followed by incubation in the absence of acoustic stimulation. In an example, methods according to the disclosure comprise exposing the sperm or a population thereof, to one or more periods of acoustic wave energy followed by incubation in the absence of acoustic stimulation. In an example, methods according to the disclosure comprise exposing the immune cell or a population thereof, to one or more periods of acoustic wave energy followed by incubation in the absence of acoustic stimulation.

In an example, the cells, or a population thereof, is exposed to acoustic wave energy once an hour for at least 2 hours. In an example, the cell or a population thereof, the ovum or a population thereof, the sperm or a population thereof, or the immune cell or a population thereof, is exposed to acoustic wave energy once an hour for at least 2 hours. In an example, the ovum or a population thereof, is exposed to acoustic wave energy once an hour for at least 2 hours. In an example, the sperm or a population thereof, is exposed to acoustic wave energy once an hour for at least 2 hours. In an example, the immune cell or a population thereof, is exposed to acoustic wave energy once an hour for at least 2 hours.

In an example, the cell or a population thereof, is exposed to acoustic wave energy once an hour for a period between about 30 seconds to about 60 minutes, for a period about 30 seconds to about 30 minutes, for a period about 5 minutes, or for a period about 10 minutes. In an example, the cell or a population thereof, is exposed to acoustic wave energy once an hour for a period between about 30 seconds to about 60 minutes. In an example, the cell or a population thereof, is exposed to acoustic wave energy once an hour for a period between about 30 seconds to about 30 minutes. In an example, the cell or a population thereof, is exposed to acoustic wave energy once an hour for a period between about 10 minutes. In an example, the cell or a population thereof, is exposed to acoustic wave energy once an hour for a period between about 5 minutes.

In an example, the cell or a population thereof, is exposed to acoustic wave energy for a period of about 5 minutes or about 10 minutes once an hour. In an example, the cell or a population thereof, is exposed to acoustic wave energy for a period of about 5 minutes once an hour. In an example, the cell or a population thereof, is exposed to acoustic wave energy for a period of about 10 minutes once an hour.

In an example, the cell or a population thereof, the ovum or a population thereof, the sperm or a population thereof, or the immune cell or a population thereof is exposed to acoustic wave energy for a period of about 5 minutes or about 10 minutes once an hour. In an example, the cell or a population thereof, the ovum or a population thereof, the sperm or a population thereof, or the immune cell or a population thereof is exposed to acoustic wave energy for a period of about 5 minutes once an hour. In an example, the cell or a population thereof, the ovum or a population thereof, the sperm or a population thereof, or the immune cell or a population thereof is exposed to acoustic wave energy for a period of about 10 minutes once an hour.

In an example, the ovum or a population thereof, is exposed to acoustic wave energy for a period of about 5 minutes or about 10 minutes once an hour. In an example, the ovum or a population thereof, is exposed to acoustic wave energy for a period of about 5 minutes once an hour. In an example, the ovum or a population thereof, is exposed to acoustic wave energy for a period of about 10 minutes once an hour.

In an example, the sperm or a population thereof, is exposed to acoustic wave energy for a period of about 5 minutes or about 10 minutes once an hour. In an example, the sperm or a population thereof, is exposed to acoustic wave energy for a period of about 5 minutes once an hour. In an example, the sperm or a population thereof, is exposed to acoustic wave energy for a period of about 10 minutes once an hour.

In an example, the immune cell or a population thereof, is exposed to acoustic wave energy for a period of about 5 minutes or about 10 minutes once an hour. In an example, the immune cell or a population thereof, is exposed to acoustic wave energy for a period of about 5 minutes once an hour. In an example, the immune cell or a population thereof, is exposed to acoustic wave energy for a period of about 10 minutes once an hour.

In an example, the cell is a mammalian cell. In an example, the cell is a human cell. In an example, the cell is a bovine cell. In an example, the cell is a marsupial cell. In an example, the cell, the ovum, the sperm, or the immune cell, is a mammalian cell. In an example, the cell, the ovum, the sperm, or the immune cell, is a human cell. In an example, the cell, the ovum, the sperm, or the immune cell, is a bovine cell. In an example, the cell, the ovum, the sperm, or the immune cell, is a marsupial cell. In an example, the ovum is a mammalian ovum. In an example, the ovum is a human ovum. In an example, the ovum is a bovine ovum. In an example, the ovum is a marsupial ovum. In an example, the sperm is a mammalian sperm. In an example, the sperm is a human sperm. In an example, the sperm is a bovine sperm. In an example, the sperm is a marsupial sperm. In an example, the immune cell is a mammalian immune cell. In an example, the immune cell is a human immune cell. In an example, the immune cell is a bovine immune cell. In an example, the immune cell is a marsupial immune cell.

In an example, the disclosure provides a cell produced using the methods disclosed herein. In an example, the disclosure provides a the cell, an ovum, a sperm, or an immune cell, produced using the methods disclosed herein. In an example, the disclosure provides an ovum produced using the methods disclosed herein. In an example, the disclosure provides a sperm produced using the methods disclosed herein. In an example, the disclosure provides an immune cell produced using the methods disclosed herein.

In an example, the disclosure provides a population of cells produced using the methods disclosed herein. In an example, the disclosure provides a population of cells, a population of ovum, a population of sperm, or a population of immune cells produced using the methods disclosed herein. In an example, the disclosure provides a population of ovum produced using the methods disclosed herein. In an example, the disclosure provides a population of sperm produced using the methods disclosed herein. In an example, the disclosure provides a population of immune cells produced using the methods disclosed herein. In an example, the population of immune cells produced using the methods disclosed herein is a population of macrophages.

In an example, the disclosure provides a method of increasing an immune response in a subject, the method comprising performing the method described herein, and administering the cell or a population thereof to the subject. In an example, the subject has a cancer, an infection, or an inflammatory disease. In an example, the subject has cancer and the cell or a population thereof expressed a CAR. In an example, the subject has cancer and the cell or population thereof is a macrophage or a population of macrophages. In this example, the macrophage or population of macrophages are differentiated from a monocyte or a population of monocytes using methods disclosed herein.

In an example, the disclosure provides a method of in vitro fertilisation, the method comprising performing the method described herein, and implanting the fertilised ovum into the reproductive tract of a female subject.

In an example, the disclosure provides a method of artificial insemination, the method comprising exposing sperm to acoustic wave energy, and implanting the sperm into the reproductive tract of a female subject.

In an example, the disclosure provides a method of differentiating a monocyte into a macrophage, the method comprising exposing the monocyte to acoustic wave energy. In an example, differentiation is characterised by one or more or all of phagocytotic ability, adherence ability, macrophage morphology, and/or surface marker expression.

### DESCRIPTION OF THE FIGURES

FIG. 1—Viability of human umbilical vein endothelial cells (HUVECs) following SRBW mechanostimulation (a) with different powers, and, (b) for different stimulation periods, relative to that of control (untreated; 0 W) cells over the same incubation period (18, 24 and 48 hrs). In (a), the SRBW exposure duration was maintained at 8 mins whereas the SRBW power was held constant at 1.5 W in (b). The viability was measured 6 hrs following SRBW exposure. Data are represented in terms of their mean value ±the standard error.

FIG. 2—(a,b) Intracellular Ca2+(n=4) and (c,d) cAMP concentration in HUVEC monolayers as function of (a,c) the applied SRBW power, and, (b,d) post-exposure incubation time, for both control (unexcited) and SRBW mechanostimulated (8 mins at 1.5 W) cells. (e) Intracellular cAMP levels in HUVEC monolayers with and without SRBW-mechanostimulation (8 mins at 1.5 W) in the presence of various Ca2+ inhibitors and chelators. (i) Ca2+ in the extracellular milieu is modulated by first incubating cells in Ca2+-free media in the absence of SRBW stimulation, followed by the addition of Ca2+ to the media, and subsequent exposure to the SRBW. (ii) The preceding case is repeated with pretreatment of the cells with an intracellular Ca2+ chelator, BAPTA-AM (BP), which chelates cytosolic Ca2+, and an ER store chelator, TPEN (TP), which chelates ER-stored Ca2+. (iii-viii) In the rest of the cases, the cells were first exposed to the SRBW (8 mins at 1.5 W), (iii) in the presence of extracellular Ca2+, following which the media is replaced with one devoid of Ca2+ but containing BP and a SERCA inhibitor, thapsigargin (TG), which prevents the influx of cytosolic Ca2+ into the ER store, prior to a final treatment with ionomycin (IM) to release Ca2+ from the ER store into the cytosol; (iv) in Ca2+-rich media, with subsequent BP and TP treatment; (v) in Ca2+-rich media, with subsequent BP and IM treatment; (vi) in Ca2+-devoid media in the presence of BP and TG; (vii) in Ca2+-devoid media, with subsequent consecutive treatment with BP and TP; and, (viii) in Ca2+ devoid media, with subsequent consecutive treatment with BP and IM. Data are represented in terms of the mean value ±the standard error over multiple runs (unless otherwise stated, experiments were carried out in triplicates (n=3)); #, † and ‡ indicate statistically significant differences with p<0.01, p<0.001 and p<0.0001, respectively.

FIG. 3—Ca2+-mediated cAMP generation in SRBW challenged cells. Upon SRBW stimulation, the cells, which originally possess intracellular Ca2+ and extracellular Ca2+ in their ground, i.e., pre-challenged, states (left), first enter into a sonochallenged condition, characterised by SF formation and the appearance of zipper-like AJs. Ca2+ from the extracellular milieu is driven into the cell as a consequence of transient membrane permeabilisation (Ramesan et al., 2018; Ambattu et al., 2020) and piezo channel activation (Liao et al., 2019; Ambattu et al., 2022), as shown by the brown arrows (centre top). The influxed Ca2+ is then rapidly transported from the cytosol into the ER store primarily through SERCA (centre bottom). Upon normalisation of cytosolic Ca2+, the ER stored Ca2+ is effluxed back into the extracellular milieu through the RyR2 receptor as shown by the green arrows (right), resulting in increased intracellular cAMP in the sonotransformed phase, which is characterised by CAB formation and stable linear AJ formation. Illustrations were created using BioRender.

FIG. 4—(a) Imaging with Fura-4AM, and, (b) quantification with Fura 2-AM, of intracellular Ca2+ in control (untreated) and SRBW mechanostimulated cells (8 mins at 1.5 W) at different post-exposure incubation times. The scale bar in (a) denotes a length of 150 μm. In (b), the SRBW mechanostimulation was carried out without and in the presence of a SERCA inhibitor (thapsigargin; TG) and a piezo channel inhibitor (ruthenium red; RR).

FIG. 5—Schematic showing the relevant pathways associated with the inhibitory studies. (a) Modulation of intracellular Ca2+ with BAPTA-AM (BP)—which chelates cytosolic Ca2+, TPEN (TP)—which chelates ER-stored Ca2+, thapsigargin (TG)—a SERCA pump inhibitor, ionomycin (IM)—an ionophore which releases Ca2+ from the ER store, and, ruthenium red (RR)—a piezo channel inhibitor. (b) Modulation of the cAMP mediated pathway using an Epac1 inhibitor HJC0197, a PKA inhibitor KT5720, a Rap1 inhibitor GGTI-298 and a Rac1 inhibitor NSC23766. Illustrations created using BioRender.

FIG. 6—(a) Epac1 expression in SRBW-mechanostimulated HUVEC monolayers (8 mins at 1.5 W) with respect to that in control (untreated) cells. (b) Linearity index, and, (c) AJ length of randomly selected SRBW mechanostimulated (8 mins at 1.5 W) cells (n=20 in (b) and n=10 in (c)) at 30 mins post exposure, in the presence of various inhibitors: HJC0197 (Epac1 inhibitor; 25 μM; HJ), KT5720 (PKA inhibitor; 10 μM; KT), GGTI-298 (Rap1 inhibitor; 15 μM; GG), NSC23766 (Rac1 inhibitor; 10 μM; NS). Data are represented in terms of the mean value±the standard error over multiple runs; ‡ indicates statistically significant differences with p<0.0001.

FIG. 7—SRBW mechanotransduction in ECs involves cytoskeletal reorganisation that traverses two phases mediated by Ca2+-cAMP signalling. An initial sonochallenge phase in which exposure of the cells to the SRBW mechanostimulation triggers an influx of Ca2+ into the cell that activates the Rho-ROCK pathway, in which actomyosin contraction gives rise to the formation of SFs and disrupted zipper-like VE-cadherin patterns that manifest as immature AJs with decreased barrier function. With further increases in intracellular Ca2+, its release from the ER store is accompanied by cAMP signalling to trigger a subsequent sonotransformation phase in which cAMP activates the Epac1-Rap1 and Rac1 pathways to not only suppress Rho-ROCK SF and immature AJ formation, but also effects the formation of CABs and stable linear AJs that lead to an enhancement in barrier function. Illustrations were created using BioRender.

FIG. 8: (a) Shows a perspective view and side view schematic of the experimental set up in which the SRBW (not to scale), generated alone a piezoelectric lithium niobate (LiNbO₃) substrate by applying an AC electric signal at the device's resonant frequency (10 MHx) to an interdigitated transducer electrode (IDT) photolithographically patterned on the substrate.

FIG. 9: (a) Oocytes were exposed SRBW with an input power of 2 W for 5 minutes and 10 minutes. The exposed oocytes are then incubated with unexposed sperm and the fertilisation rate was measured in terms of number of embryos formed. (b) sperm cells were exposed SRBW with an input power of 2 W for 5 minutes and 10 minutes. The exposed sperm cells are then incubated with unexposed oocytes and the fertilisation rate was measured in terms of number of embryos formed.

FIG. 10: Viability of Oocytes (bovine) following SRBW mechanostimulation (a) with different powers for different exposure time (minutes), relative to that of control (untreated; 0 W) cells over the same incubation period (18 hrs).

FIG. 11: cAMP concentration in stimulated sperm (bull) as function of the applied SRBW power. The cAMP was measured after 30 minutes of post-exposure incubation at different input power (W).

FIG. 12: Epac activation of Jurkat cells with SRBW stimulation and SRBW stimulated cells treated with TPEN (ER store Ca2+ chelator). Cells were exposed to 1.5 W for 10 minutes.

FIG. 13: Transformation of non-adherent to adherent cells in response to SRBW stimulation. The number of adherent cells (expressed in percentage) was determined after 36 hours of post exposure incubation. The refractory period used was 4 hours. Treatment groups are in the following order from left to right: Control, Treatment 1, Treatment 2, Treatment 3, Treatment 4, Treatment 5, Treatment 6, Treatment 7, Treatment 8.

FIG. 14: Transformation of non-adherent to adherent cells in response to SRBW stimulation: The number of adherent cells (expressed in percentage) was determined after 36 hours of post exposure incubation. The refractory period used was 2 hours. Treatment groups are in the following order from left to right: Control, Treatment 1, Treatment 2, Treatment 3, Treatment 4, Treatment 5, Treatment 6, Treatment 7, Treatment 8.

FIG. 15: Morphological changes of THP1 cells treated with SRBW stimulation for multiple days.

FIG. 16: Phagocytosis of fluorescently labelled BioParticles™ by control and SRBW stimulated cells. Phagocytosis was determined after two days of SRBW stimulation.

FIG. 17: THP-1 monocyte differentiation in macrophages. SRBW stimulated THP1 cells were fixed and immunolabelled for CD68 and CD80 followed by nuclear staining with Hoesct 33342.

FIG. 18: Cellular attachment in response to calcium inhibitors/chelators. Cells pretreated with BAPTA-AM (20 μM), Thapsigargin (300 nM) and TPEN (1 mM) were stimulated with SRBW (Treatment 4). The number of adherent cells (expressed in percentage) was determined after 36 hours of post exposure incubation.

FIG. 19: Cellular attachment in response to calcium inhibitors/chelators. Cells pretreated with GGTI-298 (15 μM) and HJC0197 (25 μM) were stimulated with SRBW. (Treatment 4). The number of adherent cells (expressed in percentage) was determined after 36 hours of post exposure incubation.

### DETAILED DESCRIPTION OF THE DISCLOSURE

### General Techniques and Selected Definitions

The term “and/or”, e.g., “X and/or Y” shall be understood to mean either “X and Y” or “X or Y” and shall be taken to provide explicit support for both meanings or for either meaning.

Reference to the singular forms “a”, “an” and “the” is also understood to imply the inclusion of plural forms unless the context dictates otherwise.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each example described herein is to be applied mutatis mutandis to each and every other example of the disclosure unless specifically stated otherwise.

Those skilled in the art will appreciate that the disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the disclosure.

The present invention as described herein can be performed using, unless otherwise indicated, conventional techniques of molecular biology, recombinant DNA technology, cell biology and immunology. Such procedures are described, for example, in Sambrook, Fritsch & Maniatis, Molecular Cloning: A Laboratory Manual, Cold Spring Harbor Laboratories, New York, Second Edition (1989), whole of vols I, II, and III; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, pp 1-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series, Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, J. eds.), vol. 2, pp. 1-284, Academic Press, New York. 12. Winsch, E., ed. (1974) Synthese von Peptiden in Houben-Weyls Metoden der Organischen Chemie (Miler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, VoIs. I-IV (D. M. Weir and C. C. Blackwell, eds., 1986, Blackwell Scientific Publications); and Animal Cell Culture: Practical Approach, Third Edition (John R. W. Masters, ed., 2000), ISBN 0199637970, whole of text.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

The terms “about”, as used herein when referring to a range is meant to encompass variations of ±20% or ±10%, more preferably ±5%, even more preferably ±1% from the specified amount.

As used herein, the term “subject” can be any animal. In one embodiment, the animal is a vertebrate. For example, the animal can be a mammal, avian, chordate, amphibian or reptile. Exemplary subjects include but are not limited to human, primate, livestock (e.g. sheep, cow, chicken, horse, donkey, pig), companion animals (e.g. dogs, cats), laboratory test animals (e.g. mice, rabbits, rats, guinea pigs, hamsters), captive wild animal (e.g. fox, deer). In one embodiment, the mammal is a human. In an embodiment, a method of the invention is for veterinary use.

As used herein, the “cell” can be from any type of subject mentioned above. The cell may have been directly obtained from a subject, have been cultured and/or may have been immortalised (i.e. be a cell line such as a hybridoma). In an embodiment, the cells are not endothelial cells. In an embodiment, the cells are not mesenchymal stem cells. In an embodiment, extracellular vesicles, such as exosomes, are not harvested from the cells. In some examples, the cell can be part of a population of cells. The term “population thereof” in the context of the present disclosure refers to a population of cells of the same type. In an example, the population of cells are a population of immune cells. In an example, the population of cells are a population of sperm cells. In an example, the population of cells are a population of ovum.

The terms “treating” or “treatment” as used herein, refer to both direct treatment of a subject by a medical professional (e.g., by administering a therapeutic agent to the subject), or indirect treatment, effected, by at least one party, (e.g., a medical doctor, a nurse, a pharmacist, or a pharmaceutical sales representative) by providing instructions, in any form, that (i) instruct a subject to self-treat according to a claimed method (e.g., self-administer a drug) or (ii) instruct a third party to treat a subject according to a claimed method. Also encompassed within the meaning of the term “treating” or “treatment” are prevention or reduction of the disease to be treated, e.g., by administering a therapeutic at a sufficiently early phase of disease to prevent or slow its progression.

Methods of the disclosure involve exposing cells to acoustic wave energy in order to increase one or more biological responses, for example increased calcium mobilisation, increased fertilisation, increased immune cell activation, and/or increased cytokine production.

As used herein, the term “increase” or “increased” means that the biological response of a cell or cell population is increased relative to a control cell or cell population that was not exposed to acoustic wave stimulation. In an example, the increase is expressed as percentage increase relative to a control. For example, the biological response is increased by between about 2% and about 1000%. In an example, the biological response is increased by about 2%, about 5%, about 10%, about 20%, about 30%, about 40%, about 50%, about 60%, about 70%, about 80%, about 90%, about 100% or greater than 100%. In an example, the biological response is increased by between about 500% and about 900%. In an example, the biological response is increased by about 900%. In an example, the biological response is increased by between about 2% and about 10%. In an example, the biological response is increased by about 50% about 150%. In an example, the biological response is increased by between about 80% and about 100%. In another example, the increase is expressed as fold-change relative to a control. In an example, the biological response is increased by between about 1.5 fold to about 4 fold. In an example, the biological response is increased by between 2 fold and about 3.5 fold. In an example, the biological response is increased by about 2 fold to about 2.5 fold.

### Calcium Mobilisation

The term “calcium mobilisation” as used herein refers to the release of internally stored calcium in cells and/or the influx of calcium from the external medium into the cell. In an example, calcium mobilisation leads to an increase in intracellular free calcium levels of in the cells.

Calcium mobilisation can be measured via intracellular calcium assays or calcium mobilisation assays that detect and measure intracellular calcium. “Intracellular calcium assay”, and “calcium mobilisation assay”, as used herein refer to cell-based assays to measure the calcium flux in a cell after acoustic wave energy exposure. Such assays utilise a calcium sensitive fluorescent dye that is taken up into the cytoplasm of most cells. The dye binds the calcium released from intracellular store and its fluorescence increases. The change in the fluorescence intensity is directly correlated to the amount of intracellular calcium that is released into cytoplasm in response to ligand activation of the receptor of interest. Examples of intracellular calcium assays include colorimetric assays and/or fluorescence-based assays using calcium probes/indicators such as Fura-2AM, Fura-4AM, Fluo-3, Fluo-4, Indo-1, Rhod-2, or Rhod-590. The labelled calcium probes/indicator can be measured by flow cytometry, spectrophotometry, and/or microscopy.

Calcium mobilisation can also be determined by measuring the upregulation of second messengers known to be involved in calcium signalling pathways. In an example, calcium mobilisation can be measured by increased intracellular cyclic AMP (cAMP).

In an example, calcium mobilisation can be measured by upregulation of exchange protein directly activated by cAMP (Epac).

The methods of increasing calcium mobilisation described herein may be used for a variety of cell types including but not limited to, immune cells, oocytes, ovum, sperm cells, and stem cells as described herein.

In an example, methods of increasing calcium mobilisation disclosed herein maintain cell viability. In example, cell viability is at least about 90% in a population of cells following exposure to acoustic wave energy and increase in calcium mobilisation. In an example, cell viability is at least about 95% in a population of cells following exposure to acoustic wave energy. In an example, cell viability is between about 80% and 95% in a population of cells following exposure to acoustic wave energy.

### Fertilisation

The present inventors have shown that exposure of ovum and/or sperm to acoustic wave energy can enhance the fertilisation process. Thus, the methods of the invention can be used to improve the likelihood of a successful fertilisation in process such as in vitro fertilisation (IVF) or artificial insemination (AI). The skilled person will appreciate that the methods disclosed herein can be applied to any form of assisted reproductive technology that involves in vitro handling of either oocytes, ova or sperm.

An ovum (or “egg”) is a mature female gamete in mammals, which gives rise to the embryo after fertilisation. Oocytes are immature ovum cells. Oocytes are developing ovum and are cells that are undergoing oogenesis. They can further be characterised into primary and secondary oocytes, depending on the meiotic stage (see, for example Alberts et al. Molecular Biology of the Cell. 4th edition. New York: Garland Science; 2002. Eggs). It is to be understood that methods of the disclosure relating to exposing ovum to acoustic wave energy encompass ovum at any stage of oogenesis (e.g. primary oocytes, secondary oocytes, mature ovum) unless otherwise specified.

A sperm cell (or “spermatozoon”) is the motile male gamete. Fertilisation occurs when a sperm penetrates an ovum to form a single celled zygote, which then develops into an embryo (a developed blastocyte, formed from cell division of the zygote). In an example, the disclosure provides a method of fertilising an ovum, the method comprising exposing an ovum to acoustic wave energy, and fertilising the ovum with a sperm. In another example, the disclosure provides a method of fertilising an ovum, the method comprising exposing sperm to acoustic wave energy, and fertilising the ovum with a sperm. In another example, the disclosure provides a method of fertilising an ovum, the method comprising exposing an ovum and a sperm to acoustic wave energy, and fertilising the ovum with a sperm.

In an example, the disclosure provides a method of in vitro fertilisation, the method comprising exposing an ovum to acoustic wave energy, and implanting the fertilised ovum into the reproductive tract of a female subject. IVF is form of assisted reproductive technology in which an ovum is fertilised with a sperm in vitro. Standard procedures for IVF are known in the art and are described, for example in Farquhar and Marjoribanks (2018), U.S. Pat. No. 7,781,207, and AU2018203649. Briefly, the process involves harvesting the ovum or ova from a female. Typically, the donor female undergoes ovarian stimulation (hyperstimulation or mild stimulation) to increase the number of ovum produced. The ovum (or ova (“eggs”)) is then retrieved from the female via techniques such as transvaginal oocyte retrieval. Transvaginal oocyte retrieval involves use of an ultrasound-guided needle piercing the vaginal wall to reach the ovaries. Through this needle, follicles can be aspirated, and the follicular fluid is examined to identify and diagnose the ova. It is common to remove between ten and thirty ovum from each patient.

The ovum (or ova) is then co-incubated with sperm in a suitable culture medium to allow fertilisation. The type of fertilisation used is often based on the male's semen parameters or other factors such as the type of analysis that may be required of the embryo. During conventional insemination, sperm are mixed with the ova in a culture dish and incubated overnight to undergo the fertilisation process. During intracytoplasmic sperm injection, one sperm is directly injected into one ovum. With either technique, the ova are examined the following day to determine whether fertilisation has occurred (e.g. cell division) using standard techniques known in the art, such as light microscopy. Methods for evaluating morphological features of fertilised ovum and pre-implantation embryos are described, for example, in Nasiri and Eftekhari-Yazdi, 2015. The fertilised ova (zygote), are then placed into specific culture media that promote growth and development of an embryo. In an example, the embryo is cultured until cleavage stage (day two to four after co-incubation). In an example, the embryo is cultured until the blastocyst stage (day five or six after co-incubation). Cultured embryos are then implanted into a female's uterus. Implantation typically occurs via catheter transfer. One or multiple embryos, for example up to 3 embryos, can be implanted.

In an example, an ovum is exposed to acoustic wave energy after retrieval and prior to co-incubation with sperm. In an example, an oocyte can be retrieved from a female and exposed to acoustic wave energy. The present disclosure contemplates that exposing an oocyte to acoustic wave energy triggers maturation into ovum. In this example, the oocyte is exposed to acoustic wave energy after retrieval and prior to co-incubation with sperm. Accordingly, in an example, the disclosure provides a method of fertilising an ovum, the method comprising exposing an oocyte to acoustic wave energy so as to produce an ovum, and fertilising the ovum with a sperm. In an example, the oocyte or ovum is exposed to acoustic wave energy using an apparatus as disclosed herein. In an example, the exposed oocytes or ovum are co-incubated with sperm that has not been exposed to acoustic wave energy. In another example, the exposed oocytes or ovum are co-incubated with sperm exposed to acoustic wave energy.

In an example, the disclosure provides a method of artificial insemination, the method comprising exposing sperm to acoustic wave energy, and implanting the sperm into the reproductive tract of a female subject. Artificial insemination is the deliberate insertion of sperm into a female subject's reproductive tract. A sperm sample is obtained (e.g. a fresh sample, or frozen sample) and prepared for insemination. Preparation generally involves thawing (if frozen), washing, and/or testing (e.g. for motility and/or blood borne diseases). The sperm is then implanted into an ovulating female (i.e. a female who has released an ovum). Implantation techniques include intracervical insemination (ICI), intrauterine insemination (IUI), or gamete intrafallopian transfer (GIFT). ICI involves injection of unwashed or raw semen into the vagina at the entrance to the cervix, usually by means of a needleless syringe. IUI involves injection of washed sperm directly into the uterus with a catheter. GIFT is another in vivo assisted reproductive technology that involves removing female's ovum, mixing the ovum with sperm and transferring the ovum and sperm into the fallopian tube via laparoscopy.

In an example, the subject is a human subject. In another example, the subject is an animal subject. In an example, the subject a non-human mammal, for example a swine, bovine, equine, canine, or feline subject. In an example, the subject is a bovine subject. In an example, the subject is a marsupial. In an example, the subject is an endangered species. In an example, the subject is a fat-tailed dunnart. Methods for assisted reproductive technologies in animals, for example in vitro fertilisation and artificial insemination as described above, follow the same basic process as human subjects. However, in some situations specialised insemination devices for particular animals can be used. Examples of suitable specialised devices are described, for example in WO199701436 and WO2010135349.

In an example, sperm is exposed to acoustic wave energy prior to insemination. In an example, sperm is exposed to acoustic wave energy using an apparatus as disclosed herein. In an example, the exposed sperm are mixed with ovum that have not been exposed to acoustic wave energy prior to insemination. In an example, the exposed sperm are mixed with ovum exposed to acoustic wave energy prior to insemination.

### Immune Cells

As used herein, the phrase “immune cell” refers to a cell which is capable of affecting or inducing an immune response upon recognition of an antigen. In some embodiments, the immune cell is a T cell, a natural killer (NK) cell, B cell, a monocyte, or a dendritic cell. In some embodiments, the cell is a mammalian cell. In some embodiments, the cell is a human cell. The cells may be autologous or allogeneic to the subject to which they are administered.

As used herein, the term “immune response” has its ordinary meaning in the art, and includes both humoral and cellular immunity. An immune response can manifest as one or more of, the development of anti-antigen antibodies, expansion of antigen-specific T cells, increase in tumor infiltrating-lymphocytes (TILs), development of an anti-tumor or anti-tumor antigen delayed-type hypersensitivity (DTH) response, clearance of the pathogen, suppression of pathogen and/or tumor growth and/or spread, tumor reduction, reduction or elimination of metastases, increased time to relapse, increased time of pathogen or tumor free survival, and increased time of survival. An immune response may be mediated by one or more of, B-cell activation, T-cell activation, natural killer cell activation, activation of antigen presenting cells (e.g., B cells, DCs, monocytes and/or macrophages), cytokine production, chemokine production, specific cell surface marker expression, in particular, expression of co-stimulatory molecules. In an example, immune cell activation is increased activity and/or cytokine production of an immune cell. In an example, the cytokine is interleukin (IL)-2, interleukin (IL)-6, tumour necrosis factor (TNF)-alpha. The immune response may be characterized by a humoral, cellular, Th1 or Th2 response, or combinations thereof. In an embodiment, the immune response is an innate immune response.

### T Cells

In some embodiments, the immune cell is a T cell e.g. a CAR-T cell. T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are several subsets of T cells, each with a distinct function.

In an embodiment, the T cells are or include central memory (TCM) T cells. TCM cells patrol lymph nodes, providing central immunosurveillance against known pathogens, but have not been described as conducting primary tissue immunosurveillance.

In an embodiment, the T cells are or include central memory stem cell (TscM) T cells. TSCM cells a rare subset of memory lymphocytes endowed with the stem cell-like ability to self-renew and the multipotent capacity to reconstitute the entire spectrum of memory and effector subset. In an embodiment, the TSCM cells include CD27⁺CD95⁺ T cells. In an example, the activated T cell is a memory T cell. In an example, methods of disclosure reactivate the memory cells which provides enhanced immune protection against pathogens, and/or prolonged immunity.

A T cell lacking a functional endogenous T cell receptor (TCR) can be, e.g., engineered such that it does not express any functional TCR on its surface, engineered such that it does not express one or more subunits that comprise a functional TCR or engineered such that it produces very little functional TCR on its surface. Alternatively, the T cell can express a substantially impaired TCR, e.g., by expression of mutated or truncated forms of one or more of the subunits of the TCR. The term “substantially impaired TCR” means that this TCR will not elicit an adverse immune reaction in a host.

A T cell described herein can be, e.g., engineered such that it does not express a functional HLA on its surface. For example, a T cell described herein, can be engineered such that cell surface expression HLA, e.g., HLA class I and/or HLA class II, is downregulated. In some embodiments, the T cell can lack a functional TCR and a functional HLA, e.g., HLA class I and/or HLA class II.

Modified T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, clustered regularly interspaced short palindromic repeats (CRISPR) transcription-activator like effector nuclease (TALEN), or zinc finger endonuclease (ZFN).

### B Cells

In some embodiments, the immune cell is a B cell. B cells, also known as B lymphocytes, are a type of white blood cell of the lymphocyte subtype. B cells function in the humoral immunity component of the adaptive immune system. B cells produce antibody molecules which may be either secreted or inserted into the plasma membrane where they serve as a part of B-cell receptors. When a naïve or memory B cell is activated by an antigen, it proliferates and differentiates into an antibody-secreting effector cell, known as a plasmablast or plasma cell. Additionally, B cells present antigens and secrete cytokines. In mammals, B cells mature in the bone marrow, which is at the core of most bones. B cells express B cell receptors (BCRs) on their cell membrane. BCRs allow the B cell to bind to a foreign antigen, against which it will initiate an antibody response.

In an example, the disclosure provides a method of activating a B cell, the method comprising exposing the cell to acoustic wave energy. In an example, B cells are activated via increased calcium signalling and/or increased calcium mobilisation. In an example, activated B cells provides enhanced immune protection against pathogens at low abundance. In example, the activated B cell is a memory B cell. In an example, activation of memory cells according to methods of the disclosure provides improved and/or prolonged immunity (e.g. decreases the need for booster doses of a vaccine). In these examples, the increased immune response is one or more of an activated B cell, enhanced immune protection against pathogens, and/or prolonged immunity.

### Natural Killer Cells

In some embodiments, the immune cell is a natural killer cell. Natural-killer (NK) cells are CD56 CD3 large granular lymphocytes that can kill infected and transformed cells, and constitute a critical cellular subset of the innate immune system. Unlike cytotoxic CD8+T lymphocytes, NK cells launch cytotoxicity against tumour cells without the requirement for prior sensitization, and can also eradicate MHC-I-negative cells. In an embodiment, the NK cells are CD3-CD56+CD7+CD127-NKp46+T-bet+Eomes+. In an embodiment, cytotoxic NK cells CD56^{dim} CD16+.

### Dendritic Cells

In some embodiments, the immune cell is a dendritic cell. Dendritic cells are a heterogeneous group of specialized antigen-presenting cells that originate in the bone marrow from CD34+ stem cells and express major histocompatibility complex (MHC) class II molecules. Mature dendritic cells are able to prime, activate and expand effector immune cells, such as T cells and NK cells. Dendritic cell therapy is known in the art (see, e.g. Sabado et al., 2017). Briefly, dendritic cells can be isolated from a patient, exposed to a disease-specific antigen, for example a cancer specific antigen, or genetically modified to express a CAR, or a disease specific antigen, and are then infused back into the patient where they prime, activate and expand effector immune cells, for example T cells.

### Monocytes

In some embodiments, the immune cell is a monocyte. Monocytes are produced in the bone marrow from monoblasts. Monocytes circulate in the bloodstream until they encounter a molecular signal that indicates damage or infection in the nearby tissue. They then migrate out of the blood into the damaged tissue. Chemotaxis of monocytes to a pathogen is controlled by multiple compounds, including monocyte chemotactic protein-1; monocyte chemotactic protein-3 (CCL7); Leukotriene B4; 5-HETE; 5-oxo-ETE; and N-Formylmethionine leucyl-phenyl al anine.

Once in a tissue, monocytes can mature into macrophages or dendritic cells. There are several subsets of monocytes in humans as defined by their surface markers, including classical (CD14⁺⁺CD16⁻), non-classical (CD14^{dim}CD16⁺⁺), and intermediate (CD14″¾ CD16⁺). While their downstream functional differences are still unclear, they each have the capacity to differentiate to macrophages under the correct stimulation conditions.

Monocytes engage in phagocytosis and cytokine production. Following opsonization by an opsonin (e.g., an antibody, complement protein, or one of several circulating proteins (e.g., pentraxins, collectins, and ficolins)) monocytes are able to engulf a pathogen. Like macrophages, monocytes are able to phagocytose pathogens by binding directly to pattern-recognition receptors on the pathogen. Monocytes also use antibody-dependent cell-mediated cytotoxicity (ADCC) to kill pathogens.

Methods of the disclosure can be used to differentiate monocytes into macrophages. Accordingly, the disclosure provides a method of differentiating a monocyte into a macrophage, the method comprising exposing the monocyte to acoustic wave energy. Monocytes can be cultured using routine techniques. In an example, monocytes can be grown in suspension in cell culture media, such as RPMI under appropriate temperature and CO2 conditions (e.g. 37° C., 5% CO2). In an example, the monocytes have been passaged at least 2, at least 3, at least 4, at least 5, at least 6, at least 8, at least 10, at least 15, or at least 20 times before exposure to acoustic wave energy. In an example, the monocytes have been passaged less than 20 times before exposure to acoustic wave energy. In an example, the monocytes have been passaged less than 15 times before exposure to acoustic wave energy. In an example, the monocytes have been passaged less than 10 times before exposure to acoustic wave energy. In an example, the monocytes have been passaged less than 7 times before exposure to acoustic wave energy. In an example, the monocytes have been passaged less than 6 times before exposure to acoustic wave energy. In an example, the monocytes have been passaged less than 5 times before exposure to acoustic wave energy.

Differentiation can be characterised by techniques known in the art and described herein. In an example, differentiation is characterised by the cell's phagocytotic ability. Phagocytotic ability can be measured by standard phagocytosis assays that measure internalization of a bacteria labelled with a fluorescent dye. In an example, differentiation is characterised by the cell's adherence ability. Monocytes are nonadherent cells whereas macrophages are adherent. Accordingly, differentiation can be determined by the number of percentage of adherent cells in culture. In an example, the percentage of adherent cells is between about 30% and 100%. In an example, the percentage of adherent cells is between about 50% and 100%. In an example, the percentage of adherent cells is between about 75% and about 95%. In an example, the percentage of adherent cells is about 95%.

Morphological features of cells can also be visualised and assessed using microscopy. Accordingly, in an example, differentiation is characterised by macrophage morphology. In an example, differentiation is characterised by the expression of macrophage surface markers. In an example, macrophages produced by methods of the disclosure express CD80 and/or CD68. In an example, macrophages produced by methods of the disclosure express CD80. In an example, macrophages produced by methods of the disclosure express CD68. In an example, macrophages produced by methods of the disclosure express CD80 and CD68. Surface markers can be measured using routine techniques in the art. For example, macrophages can be incubated with a labelled detection antibody that binds to the surface maker. The labelled detection antibody (e.g. a fluorescently labelled detection antibody) can then be visualised and quantified using flow cytometry or fluorescence microscopy.

Macrophages produced by methods according to the disclosure can be used in therapeutic applications. For example, CAR-M macrophages can be produced and administered to a subject according to the methods described herein. CAR-M macrophages are particularly useful for treating cancer. Methods of preparing and using CAR-M cells are described below.

In another example, macrophages can be artificially polarized ex vivo for adoptive transfer to a subject in need thereof. Ex vivo polarization and adoptive transfer of macrophages can be used to treat a variety of diseases, such as acute kidney injury, autoimmune encephalomyelitis, full-thickness cutaneous wounds, hepatic fibrosis caused by cystic echinococcosis, heart tissue damage, Achilles tendon rupture, spinal cord injury, and myocardial infarction.

### Chimeric Antigen Receptors

The term “chimeric antigen receptor” or alternatively “CAR” refers to a polypeptide or set of polypeptides, which when in an immune cell, provides the cell with specificity for a target cell, for example a cancer cell, and with intracellular signal generation.

CARs can be used to generate immune cells, such as T cells, dendritic cells, macrophages, or natural killer (NK) cells, specific for selected targets. Suitable constructs for generating CARs are described in U.S. Pat. Nos. 5,843,728; 5,851,828; 5,912,170; 6,004,811; 6,284,240; 6,392,013; 6,410,014; 6,753,162; 8,211,422; and WO9215322. Alternative CAR constructs can be characterized as belonging to successive generations. First-generation CARs typically consist of a single-chain variable fragment of an antibody specific for an antigen, for example comprising a VL linked to a VH of a specific antibody, linked by a flexible linker, for example by a CD8a hinge domain and a CD8a transmembrane domain, to the transmembrane and intracellular signalling domains of either CD3C or FcRy or scFv-FcRy (see, e.g., U.S. Pat. Nos. 7,741,465; 5,912,172; and 5,906,936). Second-generation CARs incorporate the intracellular domains of one or more costimulatory molecules, such as CD28, CD28z, OX40 (CD134), or 4-1BB (CD137) within the endodomain, e.g., scFv-CD28/OX40/4 BB-CD3 (see, e.g., U.S. Pat. Nos. 8,911,993; 8,916,381; 8,975,071; 9,101,584; 9,102,760; 9,102,761). Third-generation CARs include a combination of costimulatory endodomains, such a CD3C-chain, CD97, GDI la-CD18, CD2, ICOS, CD27, CD154, CDS, OX40, 4-1BB, or CD28 signalling domains, e.g., scFv-CD28-4 BB-CD3C or scFv-CD28-OX40-CD3Q (see, e.g., U.S. Pat. Nos. 8,906,682; 8,399,645; 5,686,281; WO2014134165; and WO2012079000). In some embodiments, costimulation can be coordinated by expressing CARs in antigen-specific T cells, chosen so as to be activated and expanded following, for example, interaction with antigen on professional antigen-presenting cells, with costimulation. Additional engineered receptors can be provided on the immune cells, e.g., to improve targeting of a T-cell attack and/or minimize side effects.

### Methods of Preparing CAR-Expressing Cells

### Sources of Cells

Prior to expansion, and possible genetic modification or other modification, a cell population comprising or consisting of immune cells such as monocytes, T cells, dendritic cells, natural killer (NK) cells or a combination thereof, can be obtained from a subject. Immune cells can be obtained from a number of sources, including peripheral blood mononuclear cells, bone marrow, lymph node tissue, cord blood, thymus tissue, tissue from a site of infection, ascites, pleural effusion, spleen tissue, and tumours.

In certain embodiments of the present disclosure, immune cells, e.g., T cells or monocytes, can be obtained from a unit of blood collected from a subject using any number of techniques known to the skilled artisan, such as Ficoll™ separation. In one preferred embodiment, cells from the circulating blood of an individual are obtained by apheresis. The apheresis product typically contains lymphocytes, including T cells, monocytes, granulocytes, B cells, dendritic cells, other nucleated white blood cells, red blood cells, and platelets. In one embodiment, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

It is recognized that the methods of the application can utilize culture media conditions comprising 5% or less, for example 2%, human AB serum, and employ known culture media conditions and compositions, for example those described in Smith et al. (2015).

In one embodiment, T cells are isolated from peripheral blood lymphocytes by lysing the red blood cells and depleting the monocytes, for example, by centrifugation through a PERCOLL™ gradient or by counterflow centrifugal elutriation.

The methods described herein can include, e.g., selection of a specific subpopulation of immune cells, e.g., T cells, that are a T regulatory cell-depleted population. A CD25+ depleted cell population, for example, can be obtained using, e.g., a negative selection technique, e.g., described herein. Preferably, the population of T regulatory depleted cells contains less than 30%, 25%, 20%, 15%, 10%, 5%, 4%, 3%, 2%, 1% of CD25+ cells.

In one embodiment, T regulatory (TREG) cells, e.g., CD25+ T cells, are removed from the population using an anti-CD25 antibody, or fragment thereof, or a CD25-binding ligand, IL-2. In one embodiment, the anti-CD25 antibody, or fragment thereof, or CD25-binding ligand is conjugated to a substrate, e.g., a bead, or is otherwise coated on a substrate, e.g., a bead. In one embodiment, the anti-CD25 antibody, or fragment thereof, is conjugated to a substrate as described herein.

Without wishing to be bound by a particular theory, decreasing the level of negative regulators of immune cells (e.g., decreasing the number of unwanted immune cells, e.g., TREG cells), in a subject prior to apheresis or during manufacturing of a CAR-expressing cell product can reduce the risk of subject relapse. For example, methods of depleting TREG cells are known in the art. Methods of decreasing TREG cells include, but are not limited to, cyclophosphamide, anti-GITR antibody (an anti-GITR antibody described herein), CD25−depletion, and combinations thereof.

In some embodiments, the manufacturing methods comprise reducing the number of (e.g., depleting) TREG cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete TREG cells prior to manufacturing of the CAR-expressing cell (e.g., T cell, NK cell) product.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in immune cell therapy for any number of diseases or conditions that would benefit from immune cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, Cytoxan, fludarabine, cyclosporin, FK506, rapamycin, mycophenolic acid, steroids, FR901228, and irradiation.

### Methods of Making CAR-Expressing Cells

In an embodiment, a method of the invention includes the making CAR-expressing cells by introducing a vector or nucleic acid encoding a CAR into a cell. In an example, the CAR-expressing cell is a CAR-T cell. In another example, the CAR-expressing cell is a CAR-M cell. Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art (see, for example, Sambrook Molecular Cloning: A Laboratory Manual, volumes 1-4, Cold Spring Harbor Press). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like (see, for example, U.S. Pat. Nos. 5,350,674 and 5,585,362).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

An exemplary non-viral delivery vehicle is a liposome. The use of lipid formulations is contemplated for the introduction of the nucleic acids into a host cell (in vitro, ex vivo or in vivo). In another embodiment, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma Aldrich; dicetyl phosphate (“DCP”) can be obtained from K & K Laboratories; cholesterol (“Choi”) can be obtained from Calbiochem-Behring; dimyristyl phosphatidylglycerol (“DMPG”) and other lipids may be obtained from Avanti Polar Lipids, Inc. for example. Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium.

Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

In order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### CAR-Expressing Cell Therapy

### CAR-T Cell Therapy

CAR-T cell therapy is a type of cellular therapy where immune cells (e.g., T cells, macrophages) are genetically modified to express a CAR and the CAR-expressing cell (e.g. a CAR-T cell or CAR-M cell) is infused to a recipient in need thereof. The infused cell is able to kill diseased cells expressing the target of the CAR in the recipient. Unlike antibody therapies, CAR-modified immune cells (e.g., CAR-T cells or CAR-M cells) are able to replicate in vivo resulting in long-term persistence that can lead to sustained tumour control. In various embodiments, the CAR-T cells are administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the CAR-T cell to the patient.

The invention also includes a type of cellular therapy where immune cells (e.g., T cells) are modified, e.g., by in vitro transcribed RNA, to transiently express a chimeric antigen receptor (CAR) and the CAR-T cell is infused to a recipient in need thereof. The infused cell is able to kill tumour cells in the recipient. Thus, in various embodiments, the immune cells (e.g., CAR-T cells) administered to the patient, is present for less than one month, e.g., three weeks, two weeks, one week, after administration of the CAR-T cell to the patient. Without wishing to be bound by any particular theory, the anti-tumour immunity response elicited by the CAR-T cells may be an active or a passive immune response, or alternatively may be due to a direct vs indirect immune response.

As noted above, ex vivo procedures are well known in the art and are described above. Briefly, cells are isolated from a mammal (e.g., a human) and genetically modified (i.e., transduced or transfected in vitro) with a vector expressing a CAR. The CAR-expressing cell (e.g., a CAR-T cell) can be administered to a mammalian recipient to provide a therapeutic benefit. The mammalian recipient may be a human and the CAR-expressing cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, can be applied to the cells of the present invention. Other suitable methods are known in the art, therefore the present invention is not limited to any particular method of ex vivo expansion of the cells. Briefly, ex vivo culture and expansion of immune cells (e.g., T cells) comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

The CAR-T cells or CAR-M cells of the present invention may be administered either alone, or as a pharmaceutical composition in combination with diluents and/or with other components such as IL-2 or other cytokines or cell populations, as described herein. Immune cells may be administered either alone, or as a pharmaceutical composition in combination with diluents and/or with other components such as IL-2, IL-15, or other cytokines or cell populations. Briefly, pharmaceutical compositions may comprise immune cells as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration.

A pharmaceutical composition comprising the cells described herein may be administered at a dosage of 10⁴ to 10⁹ cells/kg body weight, such as 10⁵ to 10⁶ cells/kg body weight, including all integer values within those ranges. Cell compositions may also be administered multiple times at these dosages. The cells can be administered by using infusion techniques that are commonly known in immunotherapy (see, e.g., Rosenberg et al., 1988). The optimal dosage and treatment regime for a particular patient can readily be determined by one skilled in the art of medicine by monitoring the patient for signs of disease and adjusting the treatment accordingly.

In certain embodiments, it may be desired to administer activated immune cells to a subject and then subsequently re-draw blood (or have an apheresis performed), activate and expand the immune cells therefrom, and reinfuse the patient with these activated and expanded cells. This process can be carried out multiple times every few weeks. In certain embodiments, immune cells can be activated from blood draws of from 10 cc to 400 cc. In certain embodiments, immune cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of immune cells.

### Cell Culture

Cells according to the disclosure can be cultured in a suitable culture medium according to standard cell culture techniques known in the art. The term “medium” or “media” as used in the context of the present disclosure, includes the components of the environment surrounding the cells. The media contributes to and/or provides the conditions suitable to allow cells to grow. In an example, the cell culture media can be a basal media. Examples of basal media include Minimum Essential Medium (MEM), Dulbecco's Modified Eagle's Medium (DMEM), Roswell Park Memorial Institute (RPMI), and/or any modified versions thereof.

### Methods of Culturing and Expanding Immune Cells

Immune cells such as T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and US 20060121005.

Expanding the T cells by the methods disclosed herein can multiply the cells by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers there between. In one embodiment, the T cells expand in the range of about 20 fold to about 50 fold.

In an embodiment, the cells are cultured for between about 7 days and about 14 days, or about 7 days to about 10 days.

Generally, a population of immune cells e.g., T regulatory cell depleted cells, may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For costimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besancon, France) can be used as can other methods commonly known in the art (Berg et al., 1998; Haanen et al., 1999; Garland et al., 1999).

Conditions appropriate for immune cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGF, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2). Other immune cells of the disclosure, such as monocytes and macrophages, can be cultured according to the methods described above.

### Methods of Culturing Embryos

According to the invention, the IVF-derived ovum or embryos are grown in suitable culture medium. Culture media known in the art that are suitable for use for the in vitro support of cell development and growth in laboratory procedures can be used herein. Examples include, but are not limited to human tubal fluid (HTF) (Irvine Scientific), N-2-hydroxyethylpiperazine-N′-2-ethane (HEPES) media (Irvine Scientific), IVF-50 (Scandanavian IVF Science), S2 (Scandanavian IVF Science), GI and G2 (Scandanavian IVF Science), UniIVF, ISM-1, BlastAssist, UTM media (sold as MEDICULT@media by Origio A/S), Modified Whittens medium, Wittinghams T6 media, 10 Ham's F-10 media, Earle's solution. Buffering systems, such as 4 morpholinepropanesulfonic acid (MOPS) are typically provided.

A number of specialized media or culture techniques can also be used to at varying stages of embryo development in culture. For example, specialized media can be provided for oocyte retrieval and handling; oocyte maturation; ordinary fertilisation; oocyte, zygote and embryo examination and biopsy; embryonic development to the eight-cell stage; embryonic development to the blastocyst stage; embryo transfer; and cryopreservation (see e.g. U.S. Pat. No. 6,838,235). In an example, the culture media can be specifically formulated to provide a physical environment similar to that found within the female reproductive tract and conducive to growth and development of embryos as described for example in U.S. Pat. No. 6,838,235.

Examples of specialised media include GI and G2 media which is specifically formulated to meet the physiological needs of the cleavage stage embryo and the embryo in the eight-cell through blastocyst stage of development. U.S. Pat. No. 6,605,468 discloses a medium for the propagation of early stage embryos to blastocyst stage. The medium contains an effective amount of granulocyte-macrophage colony-stimulating factor (GM-CSF) to increase the percentage of pre-blastocyst embryos which develop to transfer ready blastocysts. In an example, embryos can be co-cultured with feeder cells in a cell medium. Other stimulatory factors such as cytokines can be added to the medium to propagate embryo growth, such as leukemia inhibitory factor (LIF), see for example U.S. Pat. No. 5,418,159. Leukemia inhibitory factor is a potent hormone having general utility in the area of in vitro embryology, such as in maintaining embryonic stem cell lines and increasing the efficiency of embryo transfer.

### Culture Substrate

In an example, the cell population is cultured in a cell culture medium and seeded on a cell culture substrate prior to exposure to acoustic wave energy. Suitable culture substrates include cell culture flasks, cell culture plates, cell culture dishes, or other culture materials known in the art including glass, plastic, gels, scaffolds, or membranes.

In an example, the culture substrate is a cell culture dish, for example a petri dish.

In an example, the culture substrate is a cell culture plate.

In a cell culture flask.

In an example, the cells are non-adherent to the culture substrate.

In an example, the culture substrate is a membrane. As used herein, the term “membrane” refers to a porous substrate. Examples of suitable porous substrates include, but are not limited to, polycarbonate, poly(ε-caprolactone), polydimethylsiloxane, polyester, polystyrene, silicon nitride, and silicon dioxide. In an example, the culture substrate is polyester. In an example, the culture substrate is a Transwell plate. In an example, the culture substrate is borosilicate glass. In an example, the substrate is plastic, for example Perspex.

In an example, the culture substrate is a scaffold. As used herein, the term “scaffold” refers to a structural scaffold composed of biopolymers of the type routinely used in 3D cell culture. Examples of scaffolds include, but are not limited to, acrylamide gel, alginate gel, chitosan, solubilized basement membrane matrix gel (Matrigel), gelatin methacryloyl, cellulose, or a collagen scaffold.

In an example, the culture substrate is acrylamide gel. In an example, the acrylamide gel is a mixture of 10% (wt./vol.) acrylamide and 0.03% (wt./vol.) bisacrylamide. the acrylamide gel is a mixture of 10% (wt./vol.) acrylamide and 0.3% (wt./vol.) bisacrylamide.

In an example, the culture substrate comprises a coating to facilitate cell adhesion and/or cell growth. In an example, the culture substrate is coated with collagen. In an example, the culture substrate is coated with fibronectin. In an example, the culture substrate is coated with fibronectin. In an example, the culture substrate is coated with vitronectin. In an example, the culture substrate is coated with laminin. In an example, the culture substrate is coated with extracellular matrix proteins.

In an example, culture substrate is a porous substrate. In example, the porous substrate has a pore size of between about 0.001 μm to about 20 μm. In an example, the pore size is between about 0.003 μm and about 0.005 μm. In an example, the pore size is between about 0.3 μm and about 0.5 μm. In an example, the pore size is between about 1 μm and about 10 μm. In an example, the pore size is between about 2 μm and about 7 μm. In an example, the culture substrate comprises a pore size of about 0.4 μm.

In an example, the cells are cultured in a monolayer on the culture substrate. In an example, the culture is an organ-on-a-chip. In an example, the culture is spheroid. In an example, the culture is in a bioreactor.

Organoids are three-dimensional structures of heterogeneous tissue that function like an in vivo tissue. In other words, these three dimensional structures of tissue mimic an organ better than a traditional cell culture monolayer. Organoids therefore provide an opportunity to create cellular models of disease, which can be studied to better understand the causes of disease and identify possible treatments. Organoids are often generated from stem cells, which can be differentiated into cerebral, renal, cardiovascular and other types of organoids. Organoid technology has also been used to create a model of human colon cancer progression. These organoids may be created from normal intestinal cells mutated to transform into cancer cells, or may be derived from tumour cells per se. Organoids created from tumours have been shown to be a good reflection of the original tumour, providing opportunities for improved in vitro drug testing.

The term organoid simply means resembling an organ. Organoids are typically defined by three characteristics: self-organization, multicellularity and functionality. Thus, the cells arrange themselves in vitro into the 3-dimensional (3D) organization that is characteristic for the organ in vivo, the resulting structure consists of multiple cell types found in that particular organ and the cells execute at least some of the functions that they normally carry out in that organ. For example, a prototypical organoid, the mouse intestinal organoid, grows as a single-layered epithelium organized into domains such that it resembles the in vivo intestinal crypt-villus architecture, comprising the different cell types of the intestine (enterocytes, goblet cells, Paneth cells, enteroendocrine cells and stem cells) and surrounding a cystic lumen (Sato et al., 2009).

Organoids can be prepared from primary cultures of tissue samples. For example, organoids may be isolated from tissue samples including normal and tumour biopsies of tissues including the alimentary canal, the breast, prostate, lung, liver, ovary, pancreas, skin, kidney, brain and testis. Organoids can also be co-cultures. In an example, the organoid comprises immune cells, such as T cells, B cells, and/or macrophages. In an example, the organoid comprises reproductive cells, for example oocytes, sperm, ovary cells, or uterus cells.

Methods of culturing organoids are known in the art. Briefly, cells are obtained an seeded in a cell culture medium comprising an extracellular support matrix and cultured in a three dimensional culture under conditions described above. Other examples of culturing organoids are described, for example in WO2018011558.

### Methods of Treatment

The immune cells of or produced using the invention, e.g. CAR-T cells, are useful, inter alia, for the treatment, prevention and/or amelioration of a disease or disorder. For example, the CAR-T cells of the present invention are useful for the treatment of cancer, an infection, or an inflammatory disease. As another example, dendritic cells produced by a method of the invention can be used as a dendritic cell vaccine (see, for example, Datta et al., 2014) for treating, for example, a cancer, an infection (such as a bacterial or viral infection) or an autoimmune disease (such as diabetes). As a further example, NK cells, such as NK-CAR cells can be used to treat cancer (see, for example, Liu et al., 2021). In an further example, macrophages produced using the invention are useful for the treatment of cancer, an infection, or an inflammatory disease.

CAR-T cells may be used to treat primary and/or metastatic tumours arising in the brain and meninges, oropharynx, lung and bronchial tree, gastrointestinal tract, male and female reproductive tract, muscle, bone, skin and appendages, connective tissue, spleen, immune system, blood forming cells and bone marrow, liver and urinary tract, and special sensory organs such as the eye. In certain embodiments, CAR-T cells of the invention are used to treat one or more of the following cancers: renal cell carcinoma, pancreatic carcinoma, head and neck cancer, prostate cancer, malignant gliomas, osteosarcoma, colorectal cancer, gastric cancer (e.g., gastric cancer with MET amplification), malignant mesothelioma, multiple myeloma, ovarian cancer, small cell lung cancer, non-small cell lung cancer, synovial sarcoma, thyroid cancer, breast cancer, melanoma, leukaemia, or lymphoma.

In an embodiment, the CAR-T cells of the present invention are used to treat leukaemia, for example acute myeloid leukaemia, chronic myeloid leukaemia, acute lymphocytic leukaemia, or chronic lymphocytic leukaemia. In an embodiment, the leukaemia is acute myeloid leukaemia where low CD33+ blasts are dominant.

In another embodiment, the CAR-T cells of the present invention are used to treat lymphoma, for example Hodgkin lymphoma or non-Hodgkin lymphoma. Non-Hodgkin lymphoma types include diffuse large B-cell lymphoma, anaplastic large-cell lymphoma, Burkitt lymphoma, lymphoblastic lymphoma, mantle cell lymphoma, or peripheral T cell lymphoma. In an embodiment, the lymphoma is diffuse large B cell lymphoma or non-Hodgkin lymphoma with low levels of CD19 and/or CD20.

In the context of the methods of treatment described herein, the immune cells, such as CAR-T cells, may be administered as a monotherapy (i.e., as the only therapeutic agent) or in combination (combination therapy) with one or more additional therapeutic agents (examples of which are described elsewhere herein).

Macrophages, particularly CAR-M cells, may be also used to treat primary and/or metastatic tumours arising in the brain and meninges, oropharynx, lung and bronchial tree, gastrointestinal tract, male and female reproductive tract, muscle, bone, skin and appendages, connective tissue, spleen, immune system, blood forming cells and bone marrow, liver and urinary tract, and special sensory organs such as the eye. In certain embodiments, macrophages of the invention are used to treat one or more of the following cancers: renal cell carcinoma, pancreatic carcinoma, head and neck cancer, prostate cancer, malignant gliomas, osteosarcoma, colorectal cancer, gastric cancer (e.g., gastric cancer with MET amplification), malignant mesothelioma, multiple myeloma, ovarian cancer, small cell lung cancer, non-small cell lung cancer, synovial sarcoma, thyroid cancer, breast cancer, melanoma, leukaemia, or lymphoma.

In an embodiment, the macrophages of the present invention are used to treat leukaemia, for example acute myeloid leukaemia, chronic myeloid leukaemia, acute lymphocytic leukaemia, or chronic lymphocytic leukaemia. In an embodiment, the leukaemia is acute myeloid leukaemia where low CD33+ blasts are dominant.

In another embodiment, the macrophages of the present invention are used to treat lymphoma, for example Hodgkin lymphoma or non-Hodgkin lymphoma. Non-Hodgkin lymphoma types include diffuse large B-cell lymphoma, anaplastic large-cell lymphoma, Burkitt lymphoma, lymphoblastic lymphoma, mantle cell lymphoma, or peripheral T cell lymphoma. In an embodiment, the lymphoma is diffuse large B cell lymphoma or non-Hodgkin lymphoma with low levels of CD19 and/or CD20.

In the context of the methods of treatment described herein, the macrophages, may be administered as a monotherapy (i.e., as the only therapeutic agent) or in combination (combination therapy) with one or more additional therapeutic agents (examples of which are described elsewhere herein.

In one embodiment, the subject is at risk of developing a cancer (e.g., cancer). A subject is at risk if he or she has a higher risk of developing a cancer than a control population. The control population may include one or more subjects selected at random from the general population (e.g., matched by age, gender, race and/or ethnicity) who have not suffered from or have a family history of a cancer. A subject can be considered at risk for a cancer if a “risk factor” associated with a cancer is found to be associated with that subject. A risk factor can include any activity, trait, event or property associated with a given disorder, for example, through statistical or epidemiological studies on a population of subjects. A subject can thus be classified as being at risk for a cancer even if studies identifying the underlying risk factors did not include the subject specifically.

In one embodiment, the subject is at risk of developing a cancer and the cells, or compositions, are administered before or after the onset of symptoms of a cancer. In one embodiment, the cells, or compositions are administered before the onset of symptoms of a cancer. In one embodiment, the cells, or compositions are administered after the onset of symptoms of a cancer. In one embodiment, the cells, or compositions of the present invention is administered at a dose that alleviates or reduces one or more of the symptoms of a cancer in a subject at risk.

Examples of disease that can be treated with NK cells include, but are not limited to, cancers (e.g., melanoma, prostate cancer, breast cancer, and liver cancer) and infections, such as viral infections (e.g., infections by HSV, hepatitis viruses, human cytomegaloviruses, influenza viruses, flaviviruses, and HIV-1), bacterial infections (e.g., infections by Mycobacteria, Listeria, and Staphylococcus), and protozoan infections (e.g., infections by Plasmodium), and fungal infections (e.g., infections by Aspergillus).

As will be apparent to the skilled person a “reduction” in a symptom of a cancer in a subject will be comparative to another subject who also suffers from a cancer but who has not received treatment with a method described herein. This does not necessarily require a side-by-side comparison of two subjects. Rather population data can be relied upon. For example, a population of subjects suffering from a cancer who have not received treatment with a method described herein (optionally, a population of similar subjects to the treated subject, e.g., age, weight, race) are assessed and the mean values are compared to results of a subject or population of subjects treated with a method described herein.

### Acoustic Wave Energy

An acoustic wave (also known as “sound wave”), is a mechanical wave generated by a vibrating surface or object. Acoustic waves mainly propagate through an elastic media as longitudinal (pressure; vibration displacement parallel to the direction of wave propagation) waves that involve the compression and rarefaction of the molecules in that medium. As it is possible to support vibrations in other directions in solids, transverse (shear) waves can also exist where the vibration displacement is transverse to the direction of wave propagation.

The acoustic wavelength, is related to the frequency f by the speed at which the sound wave propagates through the medium c, which is dependent on its density and elasticity. Given this relationship and the broad spectrum of sound frequencies—ranging from infrasound (<20 Hz), audible (20 Hz-20 kHz), ultrasound (20 kHz-1 GHz) to hypersound (>1 GHz)—together with the different configurations of acoustic wave generation devices, different wave modes—i.e., the different ways acoustic waves can propagate through the media—can arise.

Types of acoustic waves include bulk acoustic waves (BAWs), surface acoustic waves (SAWs; also known as Rayleigh waves) and surface reflected bulk waves (SRBWs; also known as pseudo-SAWs) which are a hybrid surface and bulk wave. Bulk acoustic waves do not only take the form of longitudinal (pressure) or transverse (waves). Thin solid sheets with thicknesses h<λ (or h/λ<1), for example, can support plate waves that propagate parallel to its surface and through the thickness of the material. If the plate is infinitely wide, only a thickness mode exists, whereas a plate with finite width gives rise to symmetric (extensional) or asymmetric (flexural) Lamb waves that comprise both thickness and width modes. SAWs in contrast, occur in piezoelectric substrates whose thicknesses are much greater than the acoustic wavelength, i.e., h>λ, or h/λ>1, and supports a combination of both longitudinal and transverse waves. SRBWs on the other hand, can also exist in the intermediate transition regime where h≈λ, or h/λ≈1.

The coupling of sound waves to laboratory cell cultureware can be achieved with the use of an apparatus comprising an acoustic wave generator or transducer as disclosed herein. At low frequencies in the infrasound and audible range, longitudinal bulk acoustic waves can be induced in a culture chamber by coupling the piston-like vibration generated with a conventional sound transducer akin to that found in loudspeakers. To generate BAWs at ultrasound frequencies up to several MHz, piezoceramic transducers are typically used, on which electrode pads are patterned (alternatively, higher frequency (MHz) BAWs can also be generated on piezoelectric substrates). For SAWs and SRBWs, a chipscale piezoelectric substrate is employed, on which interdigitated transducers (IDTs)—electrodes patterned in an interleaved pattern—are photolithographically patterned, whose gap and spacing determines the resonant frequency of the device and hence the wavelength and frequency of the SAW or SRBW that propagates along the substrate. Whether a SRBW or SAW is generated depends on this resonant frequency and hence wavelength relative to the substrate thickness.

In an example, the acoustic wave energy is provided as bulk acoustic waves. In an example, the bulk acoustic wave is provided at a frequency of between about 1 MHz and about 10 MHz. In an example, the bulk acoustic wave is provided at a frequency of between about 1 Mhz and about 7 MHz. In an example, the bulk acoustic wave is provided at a frequency of between about 1 MHz and about 5 MHz. In an example, the bulk acoustic wave is provided at a frequency of between about 1 MHz and about 3 MHz.

In an example, the acoustic wave energy is provided as surface acoustic waves (SAWs).

In an example the acoustic wave energy is provided as surface reflected bulk waves (SRBWs). In an example, the acoustic wave energy is provided at a high frequency, i.e. greater than 1 MHz. In an example, the frequency of the acoustic energy applied to the cells is between about 1 MHz to about 1 GHz. In an example, the frequency of the acoustic energy applied to the cells is between about 1 MHz and about 50 MHz. In an example, the frequency of the acoustic energy applied to the cells is between about 1 MHz and about 30 MHz. In an example, the frequency of the acoustic energy applied to the cells is between about 5 MHz and about 20 MHz. In an example, the frequency of the acoustic energy applied to the cells is between about 5 MHz and about 15 MHz. In an example, the frequency of the acoustic energy applied to the cells is about 10 MHz.

The acoustic energy received by the cells is dependent on the input power of the acoustic wave generator as well as the dimensions, materials and configuration of the apparatus including the acoustic wave generator, the receptacle and the properties and quantity of the culture medium.

Acoustic pressure is the local pressure deviation from the ambient atmospheric pressure, caused by an acoustic wave. Acoustic pressure applied to the cells is also dependent on the input power of the acoustic wave generator as well as the dimensions, materials and configuration of the apparatus including the acoustic wave generator, the receptacle and the properties and quantity of the culture medium. In an example, cells exposed to acoustic wave energy via the apparatus of the disclosure and at a frequency of about 1 MHz to about 30 MHz are expected to be exposed to an acoustic pressure in the range of about 0.5 MPa to about 2 MPa (see, e.g. Zhang et al., 2017).

In some embodiments, the apparatus may be configured such that the acoustic energy applied to the cells by the acoustic wave generator is in the range of about 0.1 W to about 10 W, about 1 W to about 10 W, about 1 W to about 5 W, about 0.001 W to about 0.1 W, 1 mW to 50 mW, 1 mW to 10 mW, 10 mW to 100 mW, 20 mW to 80 mW, 30 mW to 60 mW, or less than 100 mW, less than 80 mW, less than 50 mW, less than 30 mW, or about 50 mW. In an example, the acoustic energy applied to the cells by the acoustic wave generator is about 2.5 W. In an example, the acoustic energy applied to the cells by the acoustic wave generator is about 2 W. In an example, the acoustic energy applied to the cells by the acoustic wave generator is about 1.5 W. The acoustic pressure applied to the cells by the acoustic wave generator may be in the range of 0.01 MPa to 1 MPa, for example. In an example, the acoustic pressure applied to the cells by the acoustic wave generator is about 0.1 MPa. In an example, an acoustic pressure applied to the cells by the acoustic wave generator is between about 0.05 MPa and about 0.5 MPa. In an example, an acoustic pressure applied to the cells by the acoustic wave generator is between about 0.08 MPa and about 0.2 MPa.

Accordingly, in an example, the cells are exposed to acoustic wave energy for a period between about 30 seconds to about 30 minutes. In an example, the cells are exposed to acoustic wave energy for a period between about 30 seconds to about 60 minutes. In an example, the cells are exposed to acoustic wave energy for a period between about 5 minutes to about 15 minutes. In an example, the cells are exposed to acoustic wave energy for a period between about 5 minutes to about 10 minutes. In an example, the cells are exposed to acoustic wave energy for a period of about 5 minutes. In an example, the cells are exposed to acoustic wave energy for a period of about 10 minutes.

In an example, the cells are immune cells and are exposed to acoustic wave energy for about 10 minutes wherein the acoustic energy applied to the cells by the acoustic wave generator is about 1.5 W. In an example, the cells are monocytes and are exposed to acoustic wave energy for about 5 minutes wherein the acoustic energy applied to the cells by the acoustic wave generator is about 1.5 W. In an example, the cells are monocytes and are exposed to acoustic wave energy for about 5 minutes wherein the acoustic energy applied to the cells by the acoustic wave generator is about 1 W. In an example, the cells are ovum, oocyte, or sperm cells and are exposed to acoustic wave energy for about 5 minutes wherein the acoustic energy applied to the cells by the acoustic wave generator is about 2 W. In an example, the cells are ovum, oocyte, or sperm cells and are exposed to acoustic wave energy for about 5 minutes wherein the acoustic energy applied to the cells by the acoustic wave generator is about 2.5 W.

Methods of the present disclosure also encompass exposing the population of cells to a period of acoustic wave energy followed by incubation in the absence of acoustic stimulation. Without wishing to be bound by theory, the present disclosure contemplates that multiple exposures to acoustic wave energy ensures that intracellular calcium levels are increased in the endoplasmic reticulum to the threshold level required to activate cAMP signalling.

In an example, the cells are exposed to one or more periods of acoustic wave energy followed by incubation in the absence of acoustic stimulation. In an example, the cells are exposed to acoustic wave energy for a short duration (e.g. about 5 minutes to about 10 minutes) followed by incubation in the absence of acoustic stimulation for one hour. In an example, the incubation in the absence of acoustic stimulation is about two hours. In an example, the incubation in the absence of acoustic stimulation is about three hours. In an example, the incubation in the absence of acoustic stimulation is about four hours. In an example, the incubation in the absence of acoustic stimulation is about five hours. In an example, the incubation in the absence of acoustic stimulation is about six hours. In an example, the incubation in the absence of acoustic stimulation is greater than about six hours.

In an example, the cycle of exposure to acoustic wave energy for a short duration followed by incubation in the absence of acoustic stimulation is repeated for at least 2 hours. In an example, the cycle of exposure to acoustic wave energy for a short duration followed by incubation in the absence of acoustic stimulation is repeated for between 2 and 6 hours. In an example, the cycle of exposure to acoustic wave energy for a short duration followed by incubation in the absence of acoustic stimulation is repeated for 6 hours. In an example, In an example, the cells are exposed to acoustic wave energy for a short duration (e.g. about 5 minutes to about 10 minutes) multiple times within an hour, for example twice an hour, or three times an hour.

Exemplary protocols for exposure of cells to acoustic wave energy according to the disclosure follow. In an example, the cells are ovum, oocyte, or sperm cells and are exposed to acoustic wave energy at a frequency of about 10 MHz, for a period of about 5 minutes, wherein the input power of the acoustic wave generator is about 2.5 W.

In an example, the cells are immune cells and are exposed to acoustic wave energy at a frequency of about 10 MHz for about 10 minutes wherein the input power of the acoustic wave generator is about 1.5 W. In an example, the cells are monocytes and are exposed to acoustic wave energy at a frequency of about 10 MHz for about 5 minutes wherein the input power of the acoustic wave generator is about 1.5 W, followed by a second exposure to acoustic wave energy at a frequency of about 10 MHz for about 5 minutes wherein the input power of the acoustic wave generator is about 1 W. In this example, a third and fourth exposure to acoustic wave energy at a frequency of about 10 MHz for about 5 minutes wherein the input power of the acoustic wave generator is about 1 W, can also be performed. In these examples, the second exposure is performed approximately one hour after the first exposure. The third exposure and fourth exposure are performed after within 24 hours after the second exposure.

### Apparatus

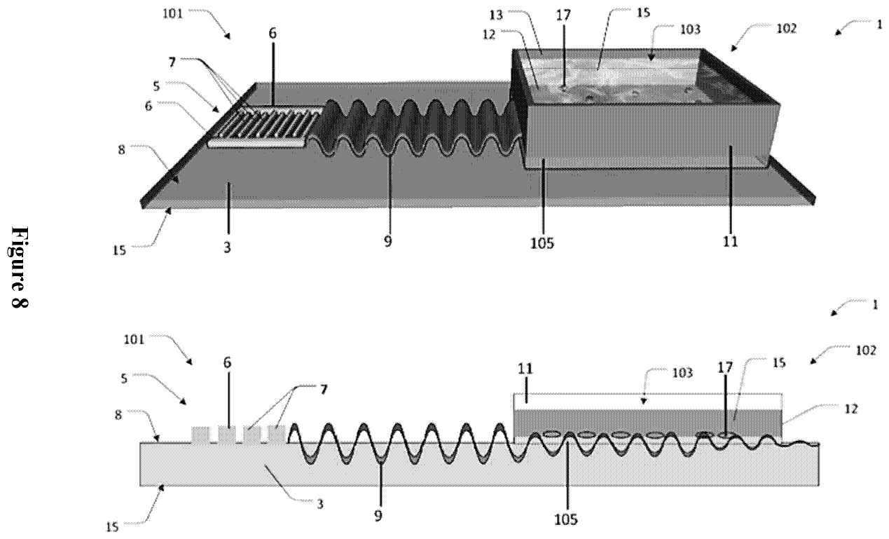

Referring to FIG. 8, an apparatus 1 for exposing a population of cells to acoustic energy (i.e., acoustic waves or vibrations) is shown, according to some embodiments.

The apparatus 1 comprises an acoustic wave generator 101 configured to generate acoustic energy at a selected power and frequency; and a receptacle 102 for accommodating a population of cells. The receptacle 102 is configured to receive acoustic energy generated by the acoustic wave generator 101.

The acoustic wave generator 101 shown in FIG. 8 comprises a piezoelectric element. However, in other embodiments, the acoustic wave generator 101 may comprise any other suitable device for generating acoustic energy or vibrations, including speakers, vibrators or other electromechanical devices.

The receptacle 102 may define a reservoir 103 configured to accommodate the population of cells in a cell medium. For example, the reservoir 103 may define a well, or in some embodiments, the reservoir may define a channel allowing the cells and culture medium to flow into and/or out of the reservoir.

The receptacle 102 may be coupled to the acoustic wave generator 101. For example, the receptacle 102 may be coupled to the acoustic wave generator 101 with a coupling material 105 to facilitate transmission of the acoustic energy from the acoustic wave generator 101 to the receptacle 102. For example, the coupling material 105 may comprise a fluid couplant, such as silicone oil.

In some embodiments, the receptacle 102 may be fixed to the acoustic wave generator 101. For example by adhesive bonding or by mechanical fastening.

In some embodiments, the receptacle 102 may be separate from the acoustic wave generator 101. For example, the receptacle 102 may not be in direct contact with the acoustic wave generator 101 during operation. The acoustic energy may be transmitted from the acoustic wave generator 101 to the receptacle 102 via a transmission medium, such as air, for example.

In some embodiments, the acoustic wave generator 101 may be disposed in direct contact with the cell medium. For example, the acoustic wave generator 101 may be at least partially submerged in the cell medium.

The apparatus 1 shown in FIG. 8 includes a piezoelectric substrate 3, for example, lithium niobate (LiNbO₃), defining a working surface 8 on which electrodes 6 of an interdigitated transducer (IDT) 5 are photolithographically patterned. The width of and gaps between the IDT fingers 7 of the electrodes 6 determine the resonant wavelength and resonant frequency of the acoustic wave generator 101.

Applying an alternating electrical signal to the IDT electrodes 6 at this resonant frequency with the aid of a signal generator and amplifier (not shown) then generates surface acoustic waves (SAW) 9 that propagate as Rayleigh waves along the working surface 8 of the substrate 3 upon which the IDT electrodes 6 are positioned. In addition to the SAW 9, surface reflected bulk waves (SRBW) can also propagate internally within the substrate 3 between the working surface 8, and an adjacent opposing surface 15 of the substrate 3. The SRBW is internally reflected between the working surface 8 and the opposing surface 15 and preferably also provides acoustic wave energy to the receptacle 102. The propagation of the SRBW may be enhanced by configuring the substrate 3 so that it has a thickness that is approximately equal to the SAW wavelength. Further description of SRBWs can be found in WO2016/179664 (RMIT University).

The receptacle 102 of the apparatus 1 of FIG. 8 is shown in the form of a well plate 11, comprising a base 12 and side walls 13 made from glass or other acoustically transmitting materials such as acrylic. The receptacle 102 is disposed on the working surface 8 of the substrate 3. The receptacle 102 defines multiple wells each configured to accommodate a population of cells 17 in cell media 15. Alternatively, the receptacle 102 may comprise one or more petri dishes, culture plates, culture flasks, transwell culture plates, microarray plates, cell flack, or other standard laboratory items for cell culture made from glass or other suitable materials. Additionally, the receptacle 102 may also comprise a fluid channel or conduit as part of a flow through system.

In some embodiments, the receptacle 102 may be integrally formed with the acoustic wave generator 101. For example, the receptacle 102 may comprise a portion of the acoustic wave generator 101, such as a portion of the substrate 3. The reservoir 103 may be defined by a recess in the working surface 8, for example.

It is also envisaged that a receptacle 102 having side walls only and no base wall could be used so that the cells and media 15 can be in direct contact with (i.e., directly coupled to) the working surface 8.

The receptacle 102 may be positioned on the work surface 8 to transmit the acoustic wave energy of the SAW 9 and preferably SRBW to the accommodated cells 17. A thin layer of silicone oil (or another fluid couplant, including water, glycerine, or other acoustic transmitting materials including gels and tapes) may be placed between the working surface 8 and base wall 12 of the well plate 11 to facilitate the coupling between the acoustic wave generator 101 and the receptacle 102, and to facilitate the transmission of the acoustic wave energy into the wells. The silicone oil may also mitigate or reduce any acoustic impedance mismatch.

In some embodiments, the acoustic wave generator 101 and substrate 3 may not be in direct contact with the receptacle 102, and may be configured to be entirely separate from the receptacle 102. For example, the acoustic wave generator 101 may be arranged to transmit acoustic energy to the cells via a transmission medium or fluid, such as a gas or liquid. The transmission medium may comprise air and/or a liquid cell medium or culture medium accommodating the cells.

In some embodiments, the substrate 3 of the acoustic wave generator 101 may be configured such that a plane of the substrate 3 is substantially perpendicular relative to a bottom plane of the receptacle. For example, the substrate 3 may be arranged substantially vertically relative to the substantially horizontal receptacle.

In an example, at least part of the acoustic wave generator is arranged in direct contact with the culture medium.

In an example, the input power for the acoustic wave generator is in the range of about 0.5 W to about 3 W. In an example, the input power for the acoustic wave generator is in the range of about 1 W to about 1.5 W. In an example, the input power for the acoustic wave generator is about 1.5 W. In an example, the input power for the acoustic wave generator is about 2 W.

In an example, the receptacle comprises a culture substrate as described herein disposed within the reservoir. In an example, the culture substrate is positioned within the reservoir so as to define a first chamber and a second chamber.

In an example, the acoustic wave generator is in direct contact with the receptacle. In an example, the acoustic wave generator is in direct contact with a tissue. In an example, the tissue is in vivo. In an example, the tissue is ex vivo. In an example, the tissue is from a mammalian subject. In an example, the tissue is an ovary. In an example, the tissue is a teste. In an example, the acoustic wave energy is provided to the cell, or a population thereof, via an apparatus, the apparatus comprising:
(i) an acoustic wave generator configured to generate acoustic energy at a selected power and frequency; and
(ii) a receptacle for accommodating a cell, or a population thereof, in a tissue, the receptacle being configured to receive acoustic energy generated by the acoustic wave generator.

In an example, the receptacle defines a reservoir configured to accommodate the cell, or a population thereof, in a tissue. In an example, at least part of the acoustic wave generator is arranged in direct contact with the tissue.

### EXAMPLES

### Example 1: Materials and Methods

### Materials

Sodium chloride, potassium chloride, magnesium chloride, methanol, ethanol, isopropanol, liquid ethane, ammonium hydroxide, sodium bicarbonate, sodium orthovanadate, disodium hydrogen phosphate, acetic acid, RNase-free water, nuclease-free water, glucose, glycerol, glycerine, non-fat skimmed milk, silicon oil, 3-isobutyl-1-methylxanthine (IBMX), dimethylsulphoxide (DMSO), β-mercaptoethanol, ethylene glycol-bis(2-aminoethylether)-N,N,N′,N′-tetraacetic acid (EGTA), Tween® 20, sodium dodecyl sulphate (SDS), Trizma® (Tris) base, phosphate buffered saline (PBS), collagen, fluorescein isothiocyanate (FITC)-labelled dextran (20, 40, 70 kDa), Tris-HCl, chloroform, ammonium persulphate (APS), acrylamide, bisacrylamide, N-hydroxysuccinimide (NHS), tetramethylethylenediamine (TEMED), fibronectin, toluene, trypsin-EDTA, formaldehyde, Triton™ X-100, bovine serum albumin (BSA), fetal bovine serum (FBS), bromophenolblue, radioimmunoprecipitation (RIPA) assay buffer, biotinylated protein ladder, phenylmethylsulfonyl fluoride (PMSF), Laemmli buffer, Hoechst 33342, ActinRed™ 555, Trypan Blue solution, bromophenol blue, Fura-2 acetoxymethyl ester (Fura-2AM), Fura-4 acetoxymethyl ester (Fura-4AM), ruthenium red, ionomycin, thapsigargin, (R)-(+)-trans-4-(1-aminoethyl)-N-(4-pyridyl)cyclohexanecarboxamide dihydrochloride (Y27632), 1,2-bis(2-aminophenoxy)ethane-N,N,N′,N′-tetraacetic acid tetrakis(acetoxymethyl ester) (BAPTA-AM), N,N,N′,N′-tetrakis(2-pyridinylmethyl)-1,2-ethanediamine) (TPEN), N6-[2-(4-diethylamino-1-methyl-butylamino)-6-methyl-pyrimidin-4-yl]-2-methyl-quinoline-4,6-diamine trihydrochloride (NSC23766), 4-cyclopentyl-2-[[(2,5-dimethylphenyl)methyl]thio]-1,6-dihydro-6-oxo-5-pyrimidinecarbonitrile (HJC0197), N-[[4-(2-(R)-amino-3-mercaptopropyl)amino]-2-naphthylbenzoyl]leucine methyl ester trifluoroacetate salt hydrate (GGTI-298), (9S,10S,12R)-2,3,9,10,11,12-hexahydro-10-hydroxy-9-methyl-1-oxo-9,12-epoxy-1H-diindolo[1,2,3-fg:3′,2′,1′-kl]pyrrolo[3,4-i][1,6]benzodiazocine-10-carboxylic acid hexyl ester (KT5720), 3-(4,5-dimethylthiazol-2-yl)-2,5-diphenyltetrazolium bromide (MTT), bicinchoninic acid (BCA) protein assay kit, Opti-MEM™ reduced serum medium, LunaScript® RT SuperMix kit, Luna® Universal qPCR Master Mix, TRiZOL™ reagent, Pierce™ ECL Western blotting detection reagent, nitrocellulose membrane (0.45 μm), protease inhibitor cocktail tablets, polyacrylamide gel, T25 cell culture flasks, Nunc™ Lab-Tek™ II Chambered Coverglass, and, 12-well polyester Transwell® inserts (0.4 μm pore size) were acquired from Thermo Fischer Scientific Pty. Ltd. (Scoresby, VIC, Australia). Cyclic AMP XP® assay, the Active Rap1 Detection Kit, anti-VE cadherin rabbit antibody, anti-GAPDH rabbit antibody, anti-rabbit IgG (H+L) F(ab′)2 fragment (Alexa Fluor® 488 conjugate), anti-biotin horse radish peroxidase (HRP)-linked antibody and anti-rabbit HRP-conjugated antibody were obtained from Cell Signalling Technology Inc. (Danvers, MA, USA). Endothelial Cell Growth Medium-2 (EGM™2) BulletKit™ was procured from Lonza Pty. Ltd. (Mount Waverley, VIC, Australia).

Human umbilical vein endothelial cells (HUVECs) were kindly supplied by Dr. Sara Baratchi (RMIT University, Bundoora, VIC, Australia), whereas human saphenous vein endothelial cells (HSaVECs), human coronary artery endothelial cells (HCAECs) and human aortic endothelial cells (HAECs) were kindly provided by Professor Yi-Chin Toh (Queensland University of Technology, Brisbane, QLD, Australia).

The following primers used for RT-qPCR analysis were acquired from Integrated DNA Technologies Inc. (Coralville, IA, USA):

GAPDH (forward):
(SEQ ID NO: 1)
5′-CATGTTCCAATATGATTCCACC-3′,
GAPDH (reverse):
(SEQ ID NO: 2)
5′-GATGGGATTTCCATTGATGAC-3′,
Epac1 (forward):
(SEQ ID NO: 3)
5′-GCAGTCCTGCTCTTTGAACC-3′,
Epac1 (reverse):
(SEQ ID NO: 4)
5′-CTGCTTGTCCACACGAAGAA-3′,
VE-cadherin (forward):
(SEQ ID NO: 5)
5′-GAACCAGATGCACATTGATGA-3′,
VE-cadherin (reverse):
(SEQ ID NO: 6)
5′-CGACTCACGCTTGACTTGATG-3′.

### Devices

The SRBW devices were fabricated as previously described (Ramesan et al., 2018; Ambattu et al., 2020; Ambattu et al., 2022). Briefly, these comprised 500-μm-thick 127.86° Y-X rotated lithium niobate (LiNbO₃) single-crystal piezoelectric substrates (Roditi Ltd., London, UK) on which 40 alternating finger pairs of 11-mm-wide and 66-nm-thick straight aluminium interdigitated transducer (IDT) electrodes in a basic full-width interleaved configuration were patterned atop a 33-nm-thick chromium adhesion layer through sputter deposition and standard ultraviolet (UV) photolithography. The SRBW wavelength, λ=398 m and thus the resonant frequency f=10 MHz of the device was set by the width and the gap of the IDT fingers (λ/4). The SRBW is then generated by applying an alternating electrical signal to the IDTs at the resonant frequency using a signal generator (SML01, Rhode & Schwarz Pty. Ltd., North Ryde, NSW, Australia) and amplifier (10 W1000C, Amplifier Research, Souderton, PA, USA). The SAW device was vertically mounted such that it is partially submerged within a well of a culture plate or the Transwell® insert.

### Cell Culture

Endothelial cells were cultured in Endothelial Cell Growth Medium-2 (EGM™2) BulletKit™ in a humidified incubator maintained at 37° C. and 5% CO2. The cells were grown in a standard T25 flask until they reached 80-90% confluency, following which they were detached using 0.05% trypsin-EDTA, reseeded in the culture plates at a density of 5000 cells/cm2 and incubated until they reached 90-95% confluency. The confluent monolayer is then exposed to the SRBW irradiation at the prescribed input power and duration. The cells were immediately processed for analysis upon removal of the acoustic field at various post-exposure incubation timepoints.

Acrylamide gels were prepared in the culture plate as previously described (Cretu et al., 2010). Briefly, acrylamide, bisacrylamide, APS, TEMED, saturated NHS solution (in toluene) and water were mixed carefully and incubated at room temperature for polymerisation. The gels were then washed thoroughly with PBS. Fibronectin crosslinking was carried out by incubating fibronectin solution (5 μg/ml in PBS) overnight at 4° C., after which it was aspirated and unreacted NHS blocked by incubating the substrate with heat inactivated (1 mg/ml) BSA solution in serum free media for 30 mins at 37° C. The BSA solution was then removed and the substrate washed thoroughly with PBS prior to cell seeding. For soft substrates, the inventors used 10% (wt./vol.) acrylamide and 0.03% (wt./vol.) bisacrylamide whereas 10% (wt./vol.) acrylamide and 0.3% (wt./vol.) bisacrylamide were used to obtain stiffer substrates.

### Cell Viability

The viability of HUVECs subject to the SRBW mechanostimulation, normalised against that of the control (untreated) cells, was assessed using an MTT proliferation assay. Briefly, the cells were incubated in MTT solution with a final concentration of 0.5 mg/ml in serum-free medium for 3 hrs at 7° C. and 5% CO2. The formazan crystals that ensued were dissolved in DMSO and the absorption measured at 570 nm using a spectrophotometric plate reader (CLARIOstar® BMG LabTech, Mornington, VIC, Australia).

### Intracellular Calcium

Free cytosolic calcium levels were determined using the fluorescent calcium indicator Fura-2AM. In brief, the EC monolayers were incubated in 5 mol/l Fura-2AM in Opti-MEM™ reduced serum medium supplemented with 2% (vol/vol) heat-inactivated FBS at 37° C. in the dark. After a 1 hr incubation period, extracellular Fura 2-AM was removed by medium change followed by a 20 min incubation period in the same medium before measurements were conducted. The fluorescence emission intensity at 510 nm in individual wells at excitation wavelengths of 340 and 380 nm were measured using a spectrophotometric plate reader (CLARIOstar® BMG LabTech, Mornington, VIC, Australia). The ratio of Fura-2 AM fluorescence emission in response to 340 nm and 380 nm excitation (340/380) was calculated and expressed in terms of the fold change with respect to that of the respective control (unexcited) cells. For imaging, Fura-4AM loaded cells were viewed under an inverted microscope (EVOS M5000, Life Technologies Corp., Bothell, WA, USA); Hoechst 33342 was used as the counterstain.

cAMP

cAMP concentrations were measured through a cyclic AMP XP™ assay. Briefly, the EC monolayers were washed in Tyrode's buffer (137mMsodium chloride, 12mMsodium bi-carbonate, 5.5 mM glucose, 2 mM potassium chloride, 1mMmagnesium chloride and 0.3 mM disodium hydrogen phosphate) at pH 7.4 supplemented with 100 mol/l IBMX—a phosphodiesterase inhibitor, to prevent the hydrolysis of cAMP. The cells were then exposed to the SRBW followed by incubation at different time points, after which the cells were lysed in 100 μl of lysis buffer that was supplied with the kit. 25 μl of the cell lysate and HRP-linked cAMP solution was subsequently transferred into the cAMP assay microtiter plates provided and incubated for 3 hrs at room temperature. Following incubation, the plate was washed four times and the liquid discarded. 100 μl of the supplied 3,3′,5,5″-tetramethylbenzidine (TMB) was then added and the solution incubated in the dark for 15 mins. The reaction was ceased by adding 100 μl of the provided STOP solution and its absorbance measured at 450 nm using a spectrophotometric plate reader (CLARIOstar, BMG LabTech, Mornington, VIC, Australia). The average of three measurements per treatment group was taken for cells from each individual culture, from which the cAMP concentrations can be calculated using a standard curve.

### Western Blotting

The SRBW challenged and untreated control cells were lysed in RIPA buffer containing 1×protease inhibitor, followed by incubation in reducing SDS loading buffer (62.5 mM Tris-HCl (pH 6.8), 2% SDS, 25% glycerol, 0.01% bromophenol blue and freshly added 5% β-mercaptoethanol) by heating at 95° C. for 5 mins. The denatured samples were then run on a 12% polyacrylamide gel and transferred onto a nitrocellulose membrane at 60 mV for 1 hr. The nitrocellulose membrane was subsequently blocked (5% non-fat skimmed milk in Tris buffered saline solution (TBST; 20 mM Tris, 150 mM sodium chloride, 0.05% Tween® 20)) for 1 hr followed by probing with primary antibody at (1:1500 dilution) overnight at 4° C. and then with secondary anti-rabbit antibody at 1:50,000 dilution for 1 hr at room temperature. The membrane was washed thrice with 1×TBST after each step. The probed membrane was then incubated in Pierce™ ECL Western blotting detection reagent at room temperature for 2 mins and subsequently visualised in a gel imager (LI-COR Biotechnology, Lincoln, NE, USA); GAPDH was used as the housekeeping gene.

### Active Rap1

Activated Rap1 was assessed using the Active Rap1 Detection Kit wherein cells were first lysed using 1× lysis buffer containing 1 mM PMSF and clarified by centrifugation (16,000×g) at 4° C. for 15 mins. An aliquot of the lysate was used to determine the amount of protein in the experimental samples by Western blotting. Clarified lysates were incubated with a glutathione S-transferase (GST) fusion protein containing the Rap1-binding domain of Ral-GDS (Ral guanine nucleotide dissociation stimulator) coupled to glutathione sepharose beads for 1 hr at 4° C. Proteins bound to the beads were then extracted in Laemmli buffer. Rap-GTP samples and total lysates (40 g) were separated in 4-20% sodium dodecyl sulphate-polyacrylamide gel electrophoresis (SDS-PAGE) gels and analysed under Western blotting against anti-Rap1 antibodies.

### RNA Isolation and Real-Time Quantitative Polymerase Chain Reaction (RT-qPCR)

Total RNA content from the control and SRBW mechanostimulated cells were isolated by homogenising the cells using TRiZOL™. The RNA-containing aqueous layer was then obtained by centrifugation of the homogenised cells in TRiZOL™ together with added chloroform. The RNA was subsequently precipitated with isopropanol and washed in ethanol, dissolved in RNAse-free water with 0.1 M EDTA and quantified using a UV spectrophotometer (NanoDrop™ One, Thermo Fisher Scientific, Waltham, MA, USA). cDNA was synthesised with the LunaScript® RT SuperMix kit and RT-qPCR carried out using the Luna® Universal qPCR Master Mix with Epac1, VE-cadherin and the aforementioned primers. GAPDH was used as housekeeping gene. As is routine in relative mRNA analysis, the relative fold change in the gene expression is normalised against that of the untreated cells.

### Statistics

Data presented in this study are expressed as the mean the standard error. Where applicable, the data was analysed using ordinary one-way analysis of variance (ANOVA) or the two-tailed, unpaired Student's t-test.

### Example 2: Calcium/cAMP-Mediated Sequential Rho-ROCK and Epac1-Rap1 Activation in Endothelial Cells

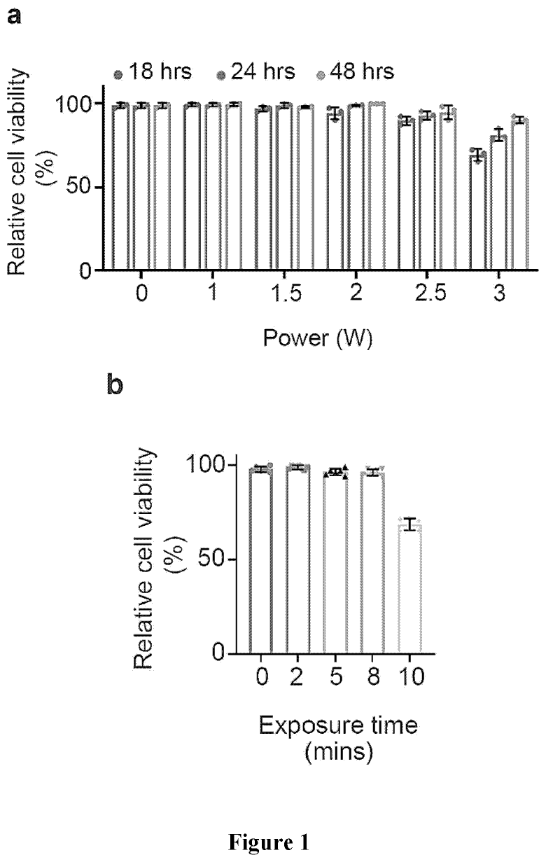

Compared to the unexposed control cells, the viability and proliferative capacity of the endothelial cells subjected to the SRBW appeared to be maintained (approximately 95%) at the lower power levels of 1 and 1.5 W, which declined to approximately 70-90% at the higher input powers of 2, 2.5 and 3 W FIG. 1(a), consistent with previous studies investigating the effect of the SRBW on epithelial cells (Ramesan et al., 2018; Ambattu et al., 2020), stem cells (Ambattu et al., 2022) and lymphoblasts (Ramesan et al., 2021).

Given that endothelial cell (EC) viability was also found to decline with longer SRBW stimulation times FIG. 1(b), the inventors employed 8 min stimulation periods in the rest of the experiments to achieve the optimal effect of the mechanostimulation while maximising cell viability.

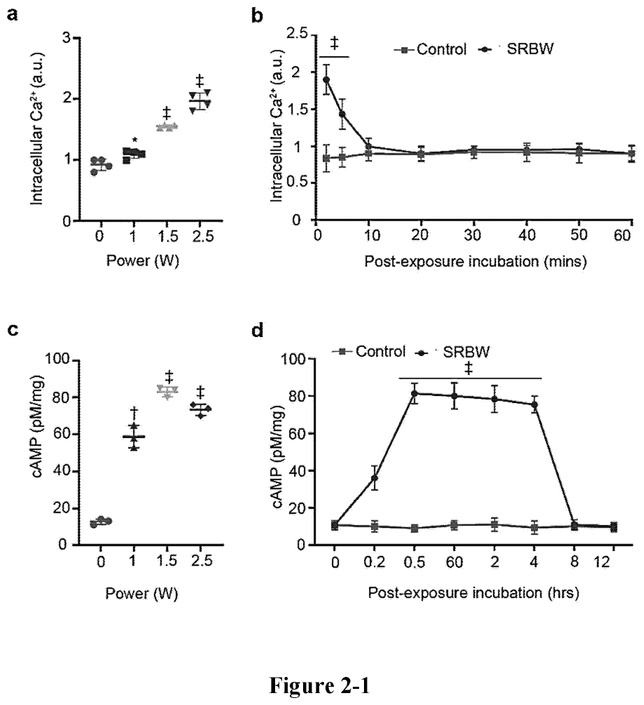

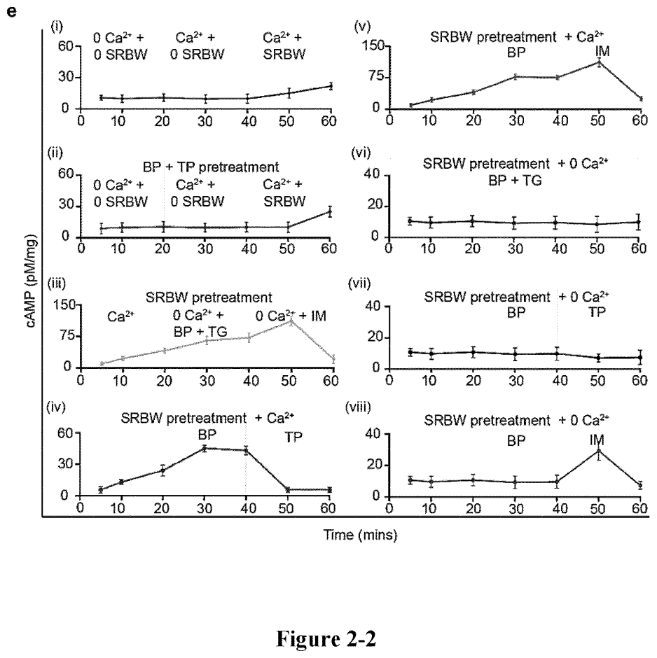

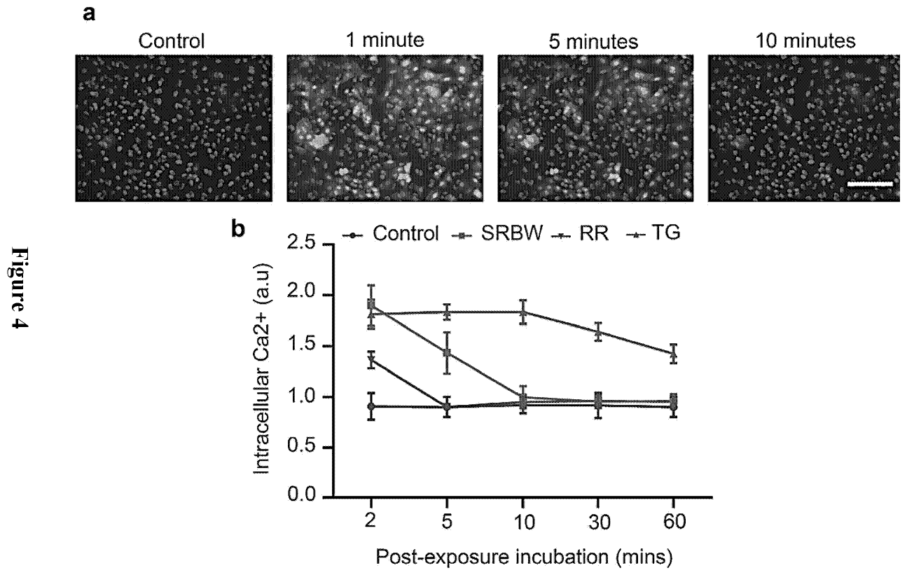

FIG. 2 and FIG. 4 show the intracellular calcium concentration as a function of the SRBW intensity and post-exposure incubation duration, respectively. The augmentation in intracellular calcium with increasing power-likely as a consequence of transient membrane permeabilisation (Ramesan et al., 2018; Ambattu et al., 2020) and piezo channel activation (Liao et al., 2019; Ambattu et al., 2022) (evident from the abatement of intracellular calcium under the high frequency excitation in the presence of a piezo channel inhibitor, ruthenium red (FIG. 4(b))—and the transient and permanent transcriptomic changes it induces being a trait that appears to be common across all SRBW mechanostimulation studies on different cell types to date (Ramesan et al., 2018; Ambattu et al., 2020; Ambattu et al., 2022). The subsequent triggering of the Rho-ROCK signalling pathway by calcium to induce SF formation and actomyosin contraction in the initial sonochallenge phase observed at early times (<0.3 hrs), confirmed by SF attenuation upon ROCK inhibition by Y27632 (data not shown), has been widely reported in the literature (Hoelzle and Svitkina, 2012; Kaunas et al., 2005; Liebner et al., 2006; Komarova and Malik, 2010; Millin et al., 2010; Huveneers et al., 2012; Dejana and Vestweber, 2013), including that on its effects on SRBW-induced mesenchymal motility and osteogenic lineage differentiation (Ambattu et al., 2022).

As a transmembrane protein that possesses both a terminal extracellular domain (ectodomain) and a terminal cytoplasmic tail, VE-cadherin is particularly sensitive to any changes in the local interendothelial microenvironment. Transient depletion of intercellular calcium, which occurs upon SRBW excitation as calcium in the extracellular milieu is transported into the cell (Ramesan et al., 2018; Femindez-Martin et al., 2012), is known to cause reversible invagination of VE-cadherin into the cell as a self-preservation mechanism against the insult (Geyer and Geyer, 1999), therefore explaining the disappearance of the immunofluorescent signal of VE-cadherin at early times (0.1 hrs) following SRBW exposure (data not shown). It is upon the slow release of calcium subsequently back into the cell to replenish intercellular calcium in the local interendothelial microenvironment that is then responsible for the restoration of the VE-cadherin signal at 0.3 hrs, whose appearance as reticulated disrupted zipper-like structures is a consequence of the large intercellular gap due to contraction of the ECs under SF tension (Tepass et al., 2000).

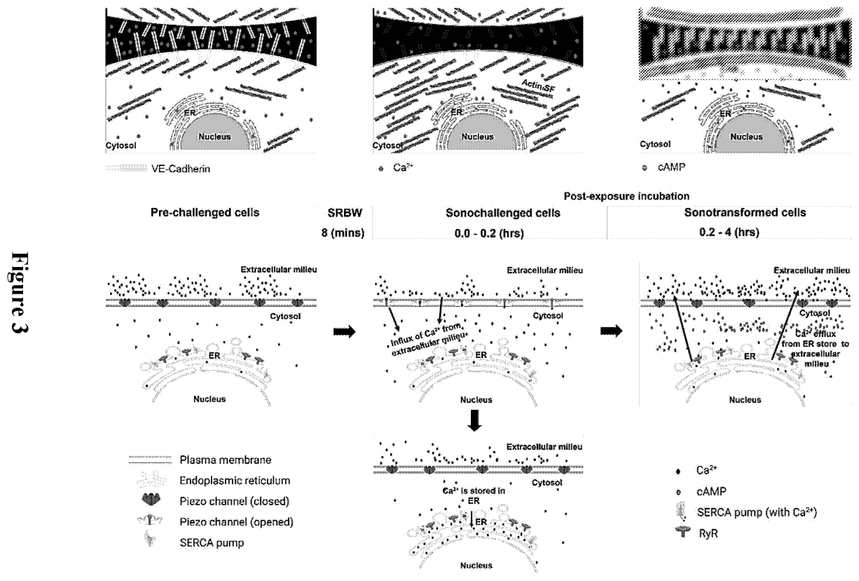

To maintain homeostasis, increases in the calcium influx into the EC due to the SRBW exposure leads to its storage in the ER, from which it is subsequently released back into the extracellular millieu; the importance of ER storage can be seen from pretreatment of the cells with the sarco/endoplasmic reticulum calcium-ATPase (SERCA) ion channel blocker thapsigargin (TG) to prevent influx of cytosolic calcium into the ER store, which lead to a deleterious effect on the cells if inhibition is prolonged for more than 0.1 hrs (FIG. 4(b)). To drive this energy-dependent calcium-induced calcium release (Roderick et al., 2003) from the ER, adenylyl cyclase (AC) is activated by the type 2 ryanodine receptor (RyR2) (Lemmens et al., 2001; Hofer et al., 2005) to rapidly convert adenosine triphosphate (ATP) into cAMP in the cytosol (Lemmens et al., 2001; Cioffi et al., 2002; Lefkimmiatis et al., 2009), as evident from the correlation between the mechanostimulated-induced calcium flux and the triggering of cAMP signalling (FIG. 3), which is dormant in the unstimulated control cells (FIG. 2(c,d)). Interestingly, despite only 8 mins of SRBW mechanostimulation and a similar period (roughly 10 mins) over which the intracellular calcium is subsequently augmented (FIG. 2/(b)), the enhancement in cAMP appears to persist for at least 4 hrs (FIG. 2(d)), which is typical of ER-depleted calcium-induced signalling (Wozniak et al., 2006).

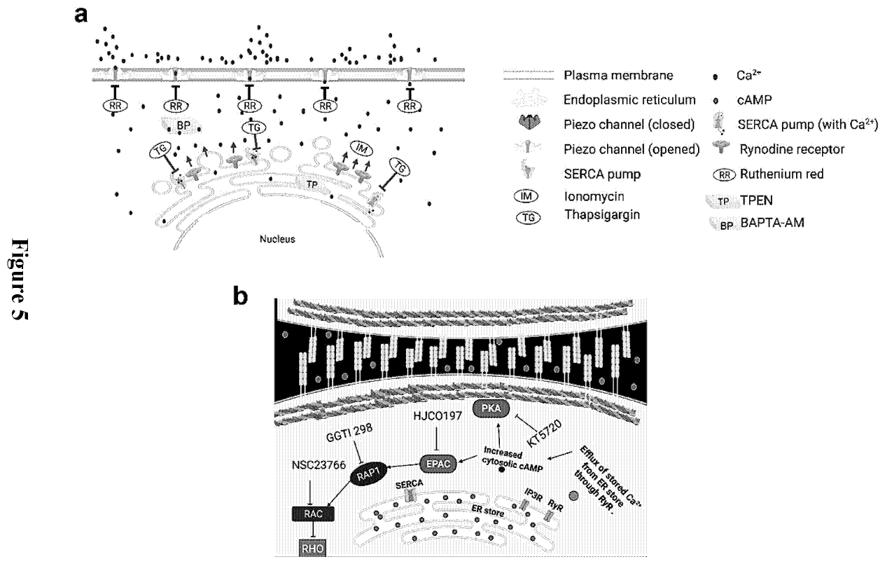

The central role of calcium release from the ER store in triggering second messenger cAMP signalling is further verified through a series of calcium inhibitory studies reported in FIG. 2(e) (see also the inhibitory pathways illustrated in FIG. 5). Foremost, the inventors note the increase in cAMP within the cells due to SRBW mechanostimulation is fairly insignificant in the absence of extracellular calcium (FIG. 2(e-i)), therefore alluding to the strong dependence of the process on extracellular calcium. Moreover, upon pretreatment with a cell-permeable intracellular calcium chelator, BAPTA-AM (BP), and an ER calcium chelator, TPEN (TP), to remove cytosolic calcium, the cAMP levels increased under the SRBW mechanostimulation only when extracellular calcium is present (FIG. 2(e-ii)). The inventors found this to be true even with SRBW pretreatment to drive extracellular calcium into the cells prior to BAPTA-AM and TPEN inhibition (FIG. 2(e-iii); 20-40 mins), suggesting the limited role of cytosolic calcium in the process. It was only upon release of calcium from ER store with the addition of ionomycin (IM) that the cAMP levels were observed to rise (FIG. 2(e-iii); 40-50 mins), indicating the critical dependence of ER-stored calcium. This is further confirmed by sequential chelation of cytosolic Calcium with BAPTA-AM and ER stored calcium with TPEN following SRBW pretreatment in the presence of extracellular Calcium in FIG. 2(e-iv) and FIG. 2(e-v). Therein, the inventors observed cAMP release due to prior influx of Calcium into the cytosol and ER as a consequence of the SRBW mechanostimulation to only be affected when Calcium in the ER store was later chelated with TPEN (FIG. 2(eiv); 40-50 mins), or, by forcing the release of calcium from the ER store with the addition of ionomycin (FIG. 2(e-v); 50-60 mins). In contrast, cAMP release was not suppressed earlier upon chelation of cytosolic calcium with BAPTA-AM (FIG. 2(e-iv) and FIG. 2(e-v); 20-40 mins). Again, the role of calcium from the ER store is dependent on sufficient transport of calcium into the ER from the extracellular milieu by the SRBW, as can be seen from the positive controls devoid of extracellular calcium in FIG. 2(e-vi)—FIG. 2(e-viii).

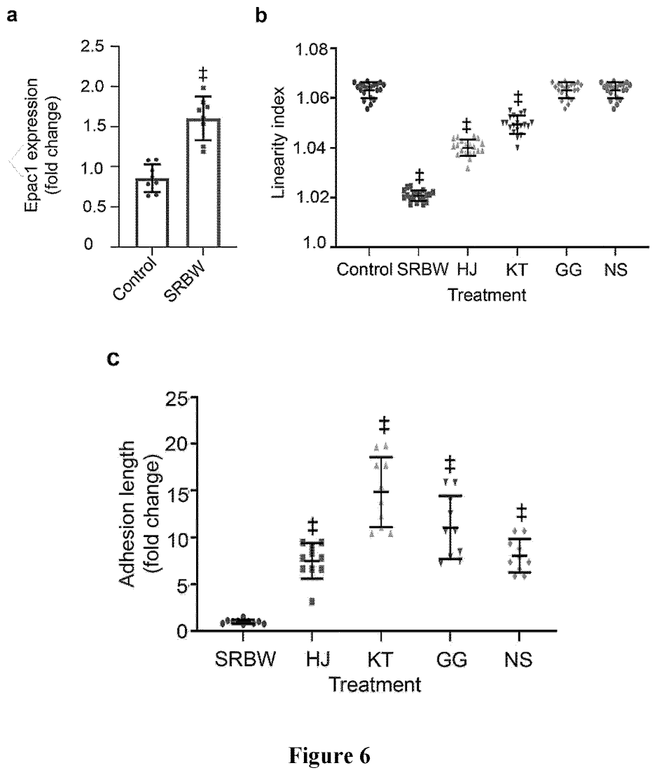

cAMP is known to maintain endothelial homeostasis through its downstream effectors protein kinase A (PKA) (Qiao et al., 2003) and Epac1 (cAMP-activated guanine nucleotide exchange protein 1) (Cheng et al., 2008; Garay et al., 2010; Bacallao 2013). In the present case, particularly the activation of the Epac1 pathway, as seen from its elevated expression in FIG. 6(a). Moreover, when treated with either an Epac1 inhibitor (HJC0197) or a PKA inhibitor (KT5720) prior to mechanostimulation the inventors observe in particular that VE-cadherin remodelling and CAB formation is quelled with the latter (data not shown), suggesting that the SRBW triggers calcium and hence cAMP signalling to activate the Epac1 pathway, which has been implicated in the suppression of Rho-ROCK-stimulated actomyosin contraction, therefore explaining the dissolution of the SFs beyond 0.3 hrs post-exposure (data not shown) when the intracellular calcium and cAMP levels are sufficiently elevated. The binding of Epac1 to cAMP also induces the activation of a plethora of diverse effectors, one of which is Rap1 (Ras-related protein 1), as can be seen from its elevated expression under SRBW mechanostimulation (data not shown)). Rap1 is known to regulate endothelial barrier function by suppressing Rho-ROCK activity and promoting stable VE-cadherin-mediated adhesion at the cellular junctions through the Rho family GTPase Rac1 (Ras-related C3 botulinum toxin substrate 1) (Ambattu et al., 2022; Liu et al., 2007; Baumer et al., 2008; Noda et al. 2010; Birukova et al, 2011; Tian et al., 2015) to induce CAB formation via CDC42-MRCK (myotonic dystrophy kinase-related CDC42-binding kinase)-mediated junctional activation of non-muscle myosin II (NMII) and CDC42-Rac1-mediated actin reorganisation (Baumer et al., 2008; Tian et al., 2015; Garcia et al., 2001; Waschke et al., 2006; Birukova et al., 2007; Knezevic et al., 2009). This role of Epac-Rap1-Rac1 in the formation of mature AJs and CABs, and hence the restoration and enhancement of endothelial barrier function, triggered by SRBW mechanostimulation is verified through the addition of Rap1 and Rac1 inhibitors, GGTI-298 and NSC23766, respectively, with which only limited CAB formation in lieu of the disrupted zipper-like VE-cadherin patterning (data not shown), which is also apparent from the departure from linearity (FIG. 6(b)) and increased adhesion site length (FIG. 6(c)).

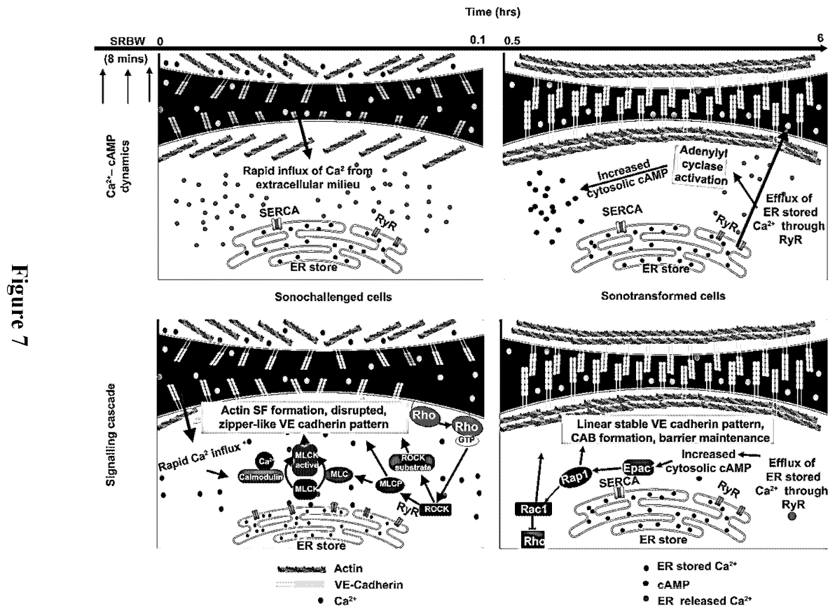

In summary, the inventors have found in this work, quite uniquely, that high frequency SRBW mechanostimulation, on its own without other stimuli, induces both the cell insult (sonochallenge) and barrier recovery (sonotransformation) phases sequentially (FIG. 7) to effect an enhancement in endothelial barrier integrity. These two phases are mediated by calcium-cAMP signalling.

### Example 3: SRBW Mediated Assisted In Vitro Fertilisation

Total fertilisation failure (TTF) occurs in 1-3% of intracytoplasmic sperm injections (ICSI). However, clinical success rates are 45% even when adequate numbers of mature oocytes are available. The inventors have discovered that SRBW can induce Ca2+ mobilisation, involving cell membrane and ER with a sustainable and slow release of Ca2+(evident from prolonged increase in intracellular cAMP). Thus, SRBW mediated assisted IVF can offer a novel protocol for addressing TTF. The SRBW can also be used to develop assisted reproductive technologies to protect endangered species like Marsupials (fat-tailed dunnart).

Intracellular cAMP

Cell were exposed SRBW and cAMP release was determined 30 minutes after SRBW treatment. cAMP concentrations were measured through a cyclic AMP XP™ assay. Briefly, the cells were washed in Tyrode's buffer (137 mM sodium chloride, 12 mM sodium bicarbonate, 5.5 mM glucose, 2 mM potassium chloride, 1 mM magnesium chloride and 0.3 mM disodium hydrogen phosphate) at pH 7.4 supplemented with 100 mol/l IBMX—a phosphodiesterase inhibitor, to prevent the hydrolysis of cAMP. The cells were then exposed to the SRBW followed by incubation at different time points, after which the cells were lysed in 100 μl of lysis buffer that was supplied with the kit. 25 μl of the cell lysate and HRP-linked cAMP solution was subsequently transferred into the cAMP assay microtiter plates provided and incubated for 3 hrs at room temperature. Following incubation, the plate was washed four times and the liquid discarded. 100 μl of the supplied 3,3′,5,5″-tetramethylbenzidine (TMB) was then added and the solution incubated in the dark for 15 mins. The reaction was ceased by adding 100 μl of the provided STOP solution and its absorbance measured at 450 nm using a spectrophotometric plate reader (CLARIOstar, BMG LabTech, Mornington, VIC, Australia). The average of three measurements per treatment group was taken for cells from each individual culture, from which the cAMP concentrations can be calculated using a standard curve.

### Oocyte Sperm Activation

Day 0: Cumulus-oocytes-complexes (COCs) were washed from maturation medium into fertilisation medium. After 22-24 hours of maturation, COCs are collected from the maturation dish and placed into a 35 mm petri dish containing VitroWash medium. COCs were then collected and washed and then transferred to a fertilisation medium containing VitroFert.

### Sperm Gradient

BoviPure gradients (at room temperature) were prepared fresh on the day of sperm preparation. A gradient top was prepared with a 40% gradient on top of the 80% gradient just prior to thawing the semen, to ensure the layers do not mix.

### Sperm Preparation

The bull semen was thawed at −35° C. for 30-60 seconds and pipetted into the gradient and centrifuged at 700 g for 20 minutes. Live sperm was carefully aspirated without disturbing the top layers and washed by centrifuging at 200 g for 5 minutes. The supernatant was removed, resuspended in fertilisation medium, and held in an incubator until required with the lid loosely capped to allow re-equilibration of media.

### SRBW Treatment

The cells (sperm/COCs or a mixture of sperm and COCs) were exposed to 10 MHz SRBW at an input power (W) of 0, 0.5, 1, 1.5, 2 and 2.5 for 5 and 10 minutes. Once exposed, cells were immediately transferred to fertilisation culture plates (Nunc″ 4-well dishes) and were incubated for appropriate time.

For cell viability studies, SRBW exposed cells (sperm or COCs) were incubated overnight at 37° C. at 5% CO2. Cell viability was measured using MTT assay. Briefly, the cells were incubated in MTT solution with a final concentration of 0.5 mg/ml in serum-free medium for 3 hrs at 7° C. and 5% CO2. The formazan crystals that ensued were dissolved in DMSO and the absorption measured at 570 nm using a spectrophotometric plate reader (CLARIOstarR BMG LabTech, Momington, VIC, Australia).

For fertilisation assessment, SRBW exposed COCs were incubated with unexposed sperm and SRBW exposed sperm were incubated with unexposed COCs in fertilisation culture plates (Nunc″ 4-well dishes) at 38.5° C., 6% CO2, 20% O2/air environment for 18-24 hours. The cumulus cells were then removed, and embryos are transferred to culture drops. The fertilisation rate is calculated by percentage of number of embryos formed from total number of COCs used for fertilisation.

### Results

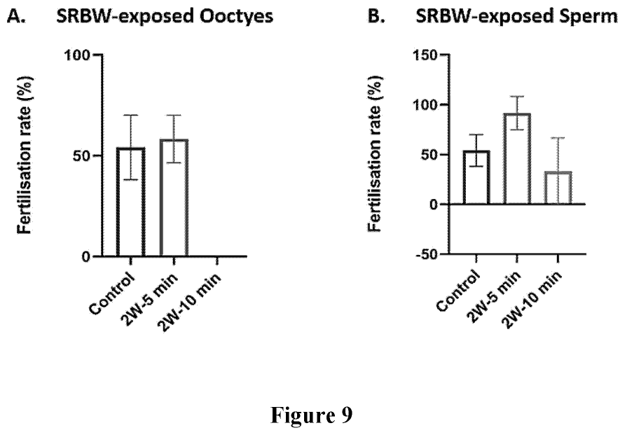

FIG. 9(a) shows oocytes that were exposed SRBW with an input power of 2 W for 5 minutes and 10 minutes. The exposed oocytes were incubated with unexposed sperm and the fertilisation rate was measured in terms of number of embryos formed. FIG. 9(a) shows that 5 minutes of SRBW exposure increased the number of embryos relative to control (no SRBW exposure).

FIG. 9(b) shows sperm cells exposed to SRBW with an input power of 2 W for 5 minutes and 10 minutes. The exposed sperm cells were then incubated with unexposed oocytes and the fertilisation rate was measured as the number of embryos formed. FIG. 9b shows that 5 minutes of SRBW exposure increased the number of embryos relative to control (no SRBW exposure) by approximately 80-90% or nearly 2-fold. The data shows that almost 100% of ovum were fertilised.

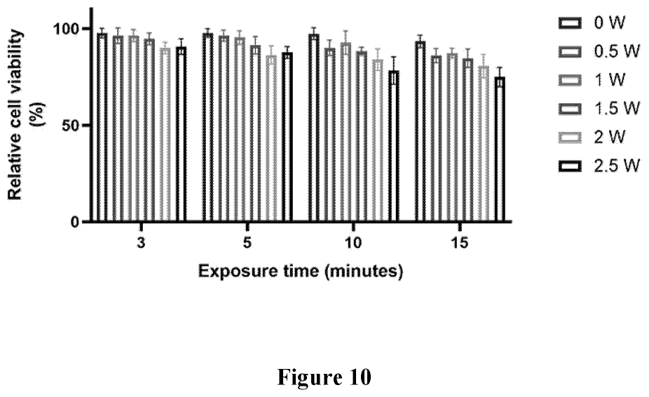

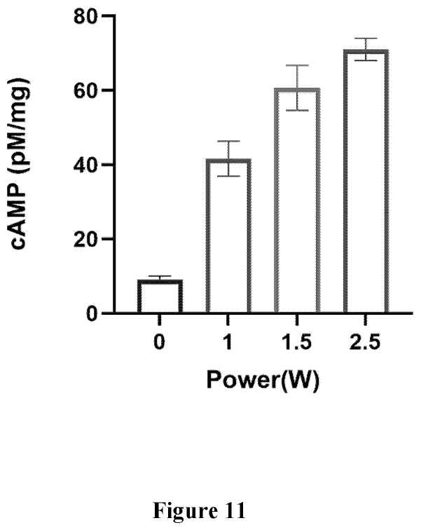

The viability of the excited sperms and oocytes was found to be greater than 90% irrespective of the input power (FIG. 10). However, as the exposure time increased the viability reduced to ˜80% as the exposure time increased. An increase in intracellular cAMP was found to occur with an increase in input power (FIG. 11). It has been noted that SRBW stimulation can increase intracellular cAMP through Ca2+ mobilisation.

These data show that exposing sperm/COCs can improve the fertilisation rate with 5 minutes exposure to 2 W input power. The fertilisation rate reduced significantly at 10 minutes exposure implicating that 10 minutes exposure may be detrimental to the embryo formation. Although, increased cAMP owing to increased Ca2+ mobilisation may be detrimental to bovine IVF, depending on the species (e.g. marsupials) these longer exposure time can be beneficial.

### Example 4: SRBW Increases EPAC Gene Activation in T Cells

### Methods

Jurkat cells (a model of T cells) were stimulated with SRBW at 10 MHz, 1.5 W for 10 minutes in the presence or absence of TPEN (ER store calcium chelator). RNA was then isolated and activation of EPAC gene was measured using real-time quantitative polymerase chain reaction (RT-qPCR) according to the method described in Example 1.

### Results

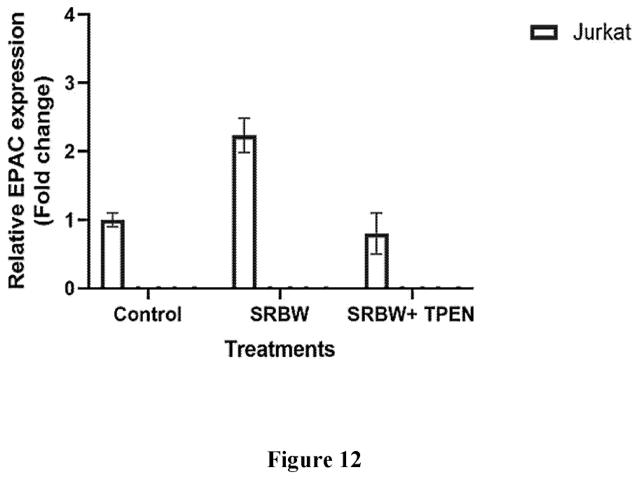

FIG. 12 shows that activation of EPAC gene in Jurkat cells is increased following SRBW exposure. These results indicate that SRBW can initiate cAMP mediated EPAC-RAP activation in T cells. Therefore, SRBW may be used for, for example, facilitating CAR-T based therapeutics development.

### Example 5: SRBW Mediated Monocyte Differentiation and Activation

Monocytes are the precursors of essential cells that control tumor progression. Monocyte-derived cells include macrophages and dendritic cells. These cells orchestrate immune reactions that determines the disease outcome and efficiency of cancer therapy. Recent anticancer therapy includes tumor-associated macrophage (TAM) polarization and functions. However, macrophages are difficult to isolate in significant quantities and proliferate in vitro for therapeutic purposes. Therefore, monocytes are a more reliable source of cells. Traditionally monocytes were differentiated using biological stimulants such as interferons, GM-CSF or chemical stimulants such as PMA and ionomycin. However, these are costly and toxic to the cells and continuous exposure brought unfavourable side effects and reductions in differentiation potential. Hence there is a need to find an alternative system to differentiate monocytes to macrophages.

### Methods

Cell culture and Mechanostimulation: THP-1 cells were obtained from CellBank (Children Medical Research institute, NSW) and grown in suspension in RPMI+Glutamax supplemented with 10% (v/v) heat-inactivated FBS with 1% penicillin-streptomycin in a humidified 37° C., 5% CO2 incubator. Low passage (passage 15 or less) cells were seeded at cell density ranging from 0.5×10⁵-0.5×10⁶ per cm². The cells were then stimulated with SRBW at a frequency of 10 Mhz and at the indicated input power and duration. The cells were stimulated and processed at different post-exposure incubation times (24 h, 36 h, 48 h, 72 h, Day 5, Day 7, Day 9).

Wash Assay: Control (untreated) and treated cells were initially characterised by wash-assay or adherence assay. Undifferentiated THP1 cells were non-adherent cells. Accordingly, cells were initially characterised by determining the percentage of adherent cells. The spent media from control and treated wells were carefully collected into labelled centrifuge tubes. The wells were washed three times with media. After each wash, the wash media was collected into corresponding centrifuge tubes. The tubes were then centrifuged at 250×g for 5 minutes. The supernatant was carefully removed, and the pellet was homogeneously mixed with 1×PBS. The cells collected from spent and wash media were counted trypan blue exclusion assay using a hemocytometer. The percentage of adherent cells was calculated by (number of cells from control wells—number of cells from treated wells/number of cells from control wells)×100. The viability was also determined using the same. The change in the cellular morphology was also recorded using Nikon.

Vybrant Phagocytosis assay: Phagocyototic ability of the cells was measured using Vybrant Phagocytotic assay as per manufacturer's instructions. Briefly, cells were seeded onto 24-well glass-bottom plate (control and treated) and were incubated with Bioparticle suspension for two hours. After the cessation of the incubation period, the BioParticles™ were removed. Trypan Blue was added for 1 minute to quench extracellular fluorescence. Following Trypan Blue aspiration, fluorescence was measured (485 nm excitation/538 nm emission) using a spectrophotometric plate reader (CLARIOstar® BMG LabTech, Mornington, VIC, Australia). Each experiment was performed with six replicate samples and repeated three times.

Immunofluorescence: The cells to be fixed were incubated in 4% formaldehyde for 20 min at room temperature followed by washing in PBS three times. This was followed by permeabilization with 0.3% Triton X for 5 min, and then blocked by incubating them in 5% BSA in PBS for 1 hour with PBS washing between each step. Following blocking, cells were incubated with primary antibodies for overnight at 4° C. For membrane markers such as anti-CD 14 (1:200), and anti-CD163 (1:200), the permeabilisation step was not included. However for anti-CD71 (1:200), anti-CD80 (1:200), anti-CD83 (1:200) and anti-CD68 (1:200), permeabilization was conducted. Following washing in PBS thrice, the cells were incubated in the secondary antibody (1:1000) for 1 hour at room temperature in the dark and subsequently washed three times in PBS. Nuclei were counterstained using Hoescht 33422 before imaging with confocal microscopy (A1 HD25, Nikon Instruments Inc., Melville, NY, USA).

### Results: Passage Number Determines the Refractory Period

One of the distinct characterisations of the monocyte differentiation is the adherence of inherently non-adherent monocyte. In this study the monocyte cell line used was THP1 cells. The preliminary characterisation of monocyte differentiation was determining the number of cells adhered after SRBW stimulation.

TABLE 1
Acoustic wave treatment parameters

| Treatment No: | First set (S₁) | Second set (S₂) |
|---|---|---|
| Treatment 1 | 0.5 W for 8 minutes | 1.5 W for 3 minutes |
| Treatment 2 | 1 W for 5 minutes | 1.5 W for 3 minutes |
| Treatment 3 | 1.5 W for 3 minutes | 0.5 W for 3 minutes |
| Treatment 4 | 1.5 W for 5 minutes | 1 W for 5 minutes |
| Treatment 5 | 1.5 W for 8 minutes | 0.5 W for 3 minutes |
| Treatment 6 | 1.5 W for 10 minutes | 1 W for 5 minutes |

Treatment consisted of two sets of treatment: the first set of treatment at particular input power for a certain amount of time, followed by a second set of stimulation, as shown in the Table 1. The second set of stimulation was done after an hour of post-exposure from the first set of stimulation. The second set of stimulation was repeated 3 times per day. Thus, a single set of SRBW stimulation included 1× of S₁ and 3× of S2.

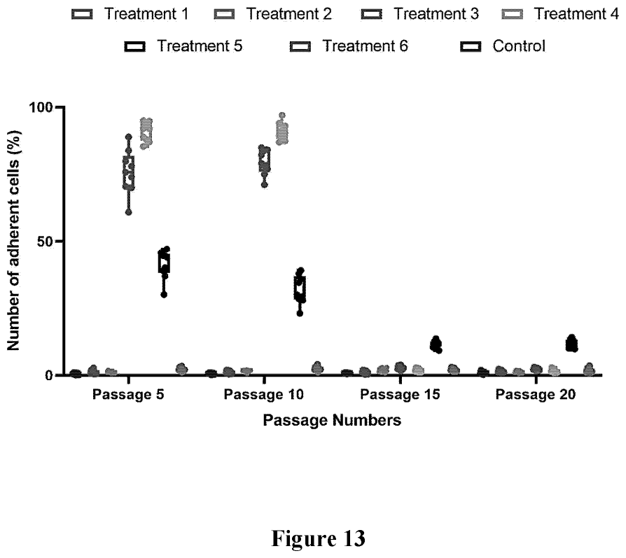

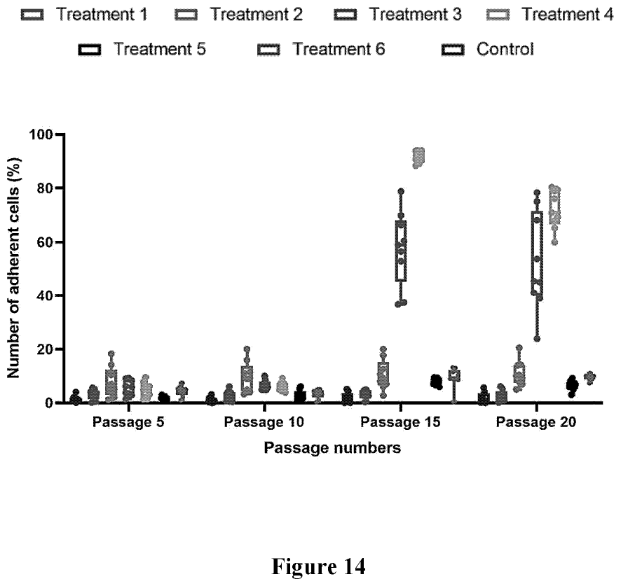

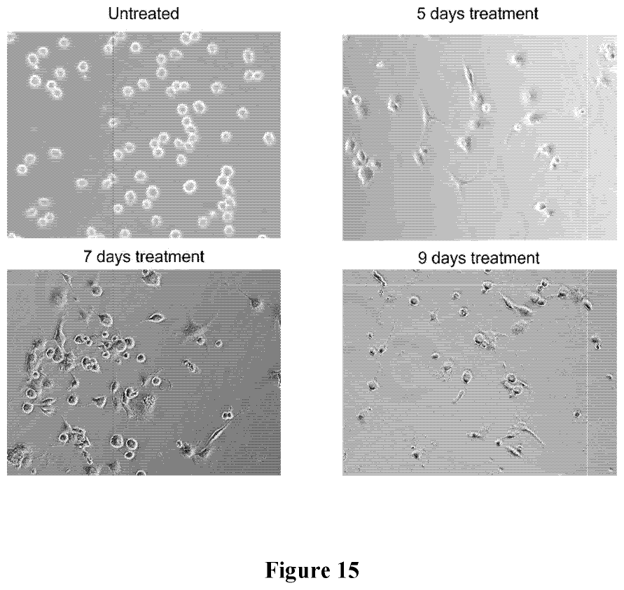

The number of cells adhered was observed after 24 hours of completion of a single set of SRBW stimulation. As shown in FIG. 13 and FIG. 14, treatment 4 showed the greatest number of adherent cells (expressed in percentage). As shown in FIG. 15, the SRBW treated cells also showed morphological distinction compared to untreated control cells. Interestingly, the interval between S₂ was found to be dependent on the passage number of the cells. For early passages P5 and P10 showed adherence with 4 hours of refractory period while for late passages, P15 and P20, the refractory period was found to be 2 hours. In addition, early passages had better responses to SRBW compared to late passages. Cells from early passages, P5 was used for most of the study unless otherwise mentioned. For the rest of the study, treatment 4 was used unless otherwise mentioned.

### SRBW Treated Cells Showed Improved Phagocytosis

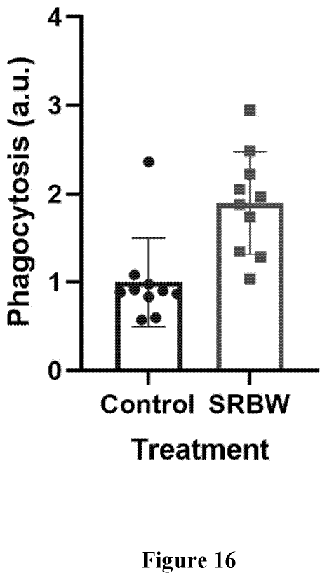

Clearance of pathogens and cell debris by monocytes and monocyte derived cells is through phagocytosis. Accordingly, phagocytotic capacity of SRBW treated cells were assessed using Vybrant assay that used fluorescently labelled BioParticles™. As shown in FIG. 16, SRBW-stimulated cells showed better phagocytosis of BioParticles™ compared to untreated THP1.

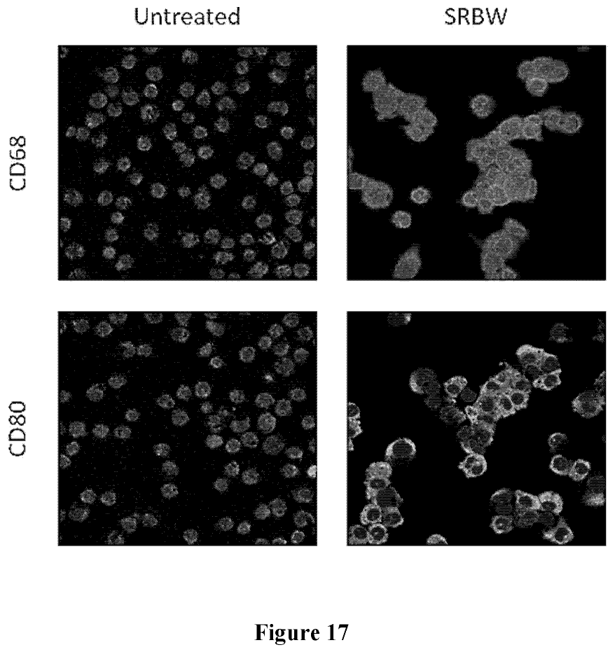

SRBW treated cells express macrophage surface markers SRBW stimulated THP1 cells were fixed and immunolabelled for CD68 (displayed in red) and CD80 (displayed in green) followed by nuclear staining Hoesct 33342. As shown in FIG. 17, SRBW-stimulated THP1 cells express both CD68 and CD80.

SRBW Facilitates Transformation of Non-Adherent Cells to Adherent Cells Through Ca2+-cAMP Mediated EPAC1-RAP1 Pathway

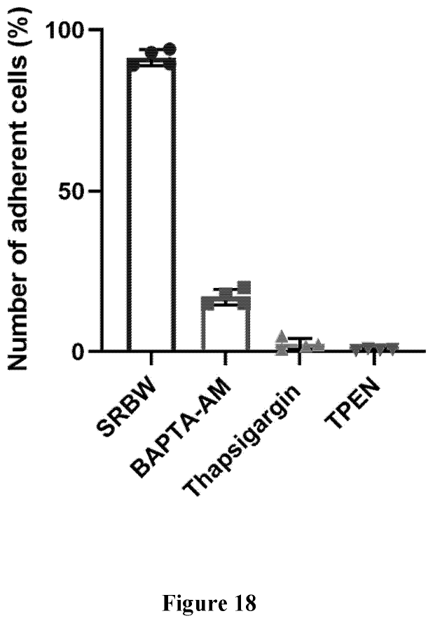

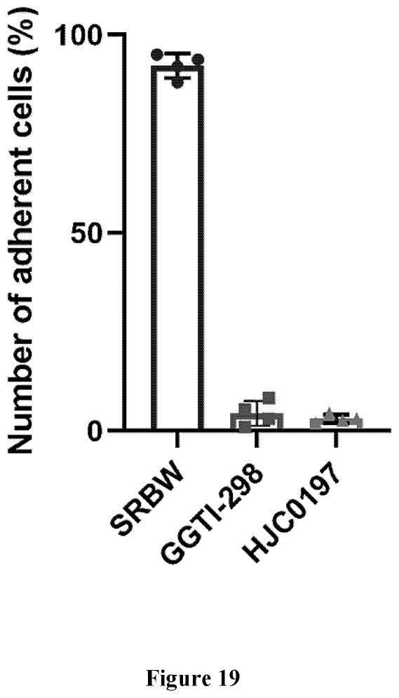

SRBW is known to increase intracellular Ca2+ thereby activating cAMP signalling which involves the EPAC1-RAP1 pathway. FIG. 18 depicts the involvement of intracellular Ca2+ in SRBW stimulated THP1 cells. In the presence of intracellular Ca2+ chelator (BAPTA-AM), the number of adherent cells reduced significantly (FIG. 19). A similar trend was seen in SRBW stimulated cells pre-treated with the SERCA-inhibitor BAPTA-AM. FIG. 19 shows that the number of adherent cells were also significantly reduced when treated with calcium chelators GGTI-298 (Rac-1 inhibitor) and HJC0197 (Epac1 inhibitor).

TPEN chelates Ca2+ in endoplasmic reticulum (ER). Non-adherent cells treated with TPEN and stimulated with SRBW failed to transform to adherent cells, thereby suggesting the involvement of cAMP in monocyte differentiation. This also suggests that multiple SRBW treatments per day are beneficial for monocyte differentiation. The magnitude of SRBW-induced increases in intracellular Ca2+ should facilitate calcium-induced calcium release from the ER, which in turn leads to the initiation of cAMP signalling. Without wishing to be bound by a particular theory, the results suggest that it may beneficial for mechanostimulation to enhance intracellular Ca2+ to the level required for initiating cAMP signalling. This could also be reason why cells from different passage number respond to different treatments for differentiation.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

All publications discussed above are incorporated herein in their entirety. Any discussion of documents, acts, materials, devices, articles or the like which has been included in the present specification is solely for the purpose of providing a context for the present disclosure. It is not to be taken as an admission that any or all of these matters form part of the prior art base or were common general knowledge in the field relevant to the present disclosure as it existed before the priority date of each claim of this application.

### REFERENCES

Alberts et al. (2020). Molecular Biology of the Cell. 4th edition. Eggs
Ambattu et al. (2020) Communications Biology 3: 1-9.
Ambattu et al. (2022) Small 18: 2106823.
Bacallao et al. (2013) PLoS One 8: e82354.
Baumer et al. (2008) Histochemistry and Cell Biology 129: 765-778.
Berg et al. (1998) Transplant Proc. 30:3975-3977.
Birukova et al. (2011) Experimental Cell Research 317: 859-872.
Birukova et al. (2007) Experimental Cell Research 313: 2504-2520.
Cheng et al. (2008) Acta Biochimica et Biophysica Sinica 40: 651-662.
Cioffi et al. (2002) Journal of Cell Biology 157: 1267-1278.
Cretu et al. (2010) Journal of Visualized Experiments 42:e2089.
Datta et al. (2014) Yale J Biol Med 87:491-518.
Dejana and Vestweber (2013) Progress in Molecular Biology and Translational Science 116: 119-144.
Farquhar and Marjoribanks (2018) Cochrane Database Sys Rev 17:CD010537.
Fernindez-Martin et al. (2012) Arteriosclerosis, Thrombosis, and Vascular Biology 32: e90-e102.
Garay et al. (2010) Journal of Immunology 185: 3227-3238.
Garcia et al. (2001) Journal of Clinical Investigation 108: 689-701.
Garland et al. (1999) J. Immunol Meth. 227:53-63.
Geyer and Geyer (1999) Glycobiology 9: 915-925.
Ghosh et al. (1991) Glycobiology 5:505-510.
Haanen et al. (1999) J. Exp. Med. 190:1319-1328.
Hoelzle and Svitkina (2012) Molecular Biology of the Cell 23: 310-323.
Hofer (2005) Journal of Cell Science 118: 855-862.
Huveneers et al. (2012) Journal of Cell Biology 196: 641-652.
Kaunas et al. (2005) Proceedings of the National Academy of Sciences 102: 15895-15900.
Knezevic et al. (2009) Journal of Biological Chemistry 284: 5381-5394.
Komarova and Malik (2010) Annual Review of Physiology 72: 463-493.
Lefkimmiatis et al. (2009) Nature Cell Biology 11: 433-442.
Lemmens et al. (2001) Journal of Biological Chemistry 276: 9971-9977.
Liao et al. (2019) Biochemical and Biophysical Research Communications 518: 541-547.
Liebner et al. (2006) Thrombosis, and Vascular Biology 26: 1431-1438.
Liu et al. (2007) Circulation Research 101: e44-e52.
Millin et al. (2010) BMC Biology 8: 1-13.
Nasiri and Eftekhari-Yazdi (2015) Cell J. 16(4):392-405
Noda et al. (2010) FEBS Letters 584: 1379-1385.
Qiao et al. (2003) American Journal of Physiology-Lung Cellular and Molecular
Physiology 284: L972-L980.
Ramesan et al. (2018) Nanoscale 10: 13165-13178.
Ramesan et al. (2021) ACS Applied Bio Materials 4: 2781-2789.
Roderick et al. (2003) Current Biology 13: R425.
Rosenberg et al. (1988) New Eng. J. of Med 319:1676.
Sabado et al. (2017) Cell Res 27:74-95.
Sato et al. (2009) Nature 459:262-265.
Smith et al. (2015) Clinical & Translational Immunology 4:e31.
Tepass et al. (2000) Nature Reviews Molecular Cell Biology 1: 91-100.
Tian et al. (2015) Molecular Biology of the Cell 26: 636-650.
Waschke et al. (2006) Histochemistry and Cell Biology 125: 397-406.
Wozniak et al. (2006) Journal of Cell Biology 175: 709-714.
Zhang et al. (2017) Small 13: 1602962.

1. A method of increasing calcium mobilisation in a cell comprising exposing the cell to acoustic wave energy.

2. The method of claim 1, wherein in the cell is selected from an immune cell, an ovum or sperm.

3. A method of fertilising an ovum, the method comprising
1) exposing the ovum to acoustic wave energy, and
2) fertilising the ovum with a sperm; or
1) exposing sperm to acoustic wave energy, and
2) fertilising the ovum with the sperm.

4. A method of activating an immune cell, the method comprising exposing the immune cell to acoustic wave energy.

5. A method of increasing cytokine production of an immune cell, the method comprising activating the immune cell by exposing the immune cell to acoustic wave energy.

6. The method of any one of claims 2, 4 or 5, wherein the immune cell is a T cell, B cell, dendritic cell, a monocyte, or a natural killer cell.

7. The method of any one of claims 2 or 4 to 6, wherein the immune cell comprises an exogenous chimeric antigen receptor (CAR).

8. The method of claim 7, wherein the immune cell is a CAR-T cell or a CAR-M cell.

9. The method of any one of claims 1 to 8, wherein the acoustic wave energy is provided in the form of surface acoustic waves (SAW).

10. The method of any one of claims 1 to 9, wherein the acoustic wave energy is provided in the form of surface reflected bulk waves (SRBW).

11. The method of any one of claims 1 to 8, wherein the acoustic wave energy is provided in the form of a bulk acoustic wave (BAW).

12. The method of any one of claims 1 to 11, wherein the cell, or a population thereof, are in a cell culture medium.

13. The method of any one of claims 1 to 11, wherein the cell, or a population thereof, are in a tissue.

14. The method of any one of claims 1 to 11, wherein the acoustic wave energy is provided to the cell, or a population thereof, via an apparatus, the apparatus comprising:
(i) an acoustic wave generator configured to generate acoustic energy at a selected power and frequency; and
(ii) a receptacle for accommodating a cell, or a population thereof, in a culture medium, the receptacle being configured to receive acoustic energy generated by the acoustic wave generator.

15. The method of claim 14, wherein the receptacle defines a reservoir configured to accommodate the cell, or a population thereof, in a culture medium.

16. The method of claim 14 or claim 15, wherein the acoustic wave generator comprises a piezoelectric substrate defining a working surface and an interdigitated transducer located on and in contact with the working surface of the piezoelectric substrate.

17. The method of claim 16, wherein the acoustic wave energy is propagated as a surface acoustic wave (SAW) along the working surface.

18. The method of claim 16, wherein the acoustic wave energy is propagated as a surface reflected bulk wave (SRBW) within the piezoelectric substrate and internally reflected between the working surface and an adjacent surface of the piezoelectric substrate.

19. The method of any one of claims 16 to 18, wherein at least part of the acoustic wave generator is arranged in direct contact with the culture medium.

20. The method of any one of claims 16 to 18, wherein the acoustic wave generator is separated from the culture medium.

21. The method of any one of claims 14 to 20, wherein the receptacle is coupled to the acoustic wave generator with a coupling material.

22. The method of any one of claims 1 to 21, wherein the frequency of the applied acoustic energy is in the range of about 1 MHz to about 1 GHz.

23. The method of claim 22, wherein the frequency of the applied acoustic energy is about 10 MHz.

24. The method of any one of claims 14 to 23, wherein the input power for the acoustic wave generator is in the range of about 0.5 W to about 2.5 W.

25. The method of any one of claims 14 to 24, wherein an acoustic pressure applied to the cell, or a population thereof, by the acoustic wave generator is about 0.1 MPa.

26. The method of claim 25, wherein the cell, or a population thereof, is exposed to acoustic wave energy for a period between about 30 seconds to about 60 minutes, preferably between about 30 seconds to about 30 minutes.

27. The method of claim 26, wherein the cell, or a population thereof, is exposed to acoustic wave energy for a period of about 5 minutes or about 10 minutes.

28. The method of any one of claims 1 to 27, comprising exposing the cell, or a population thereof, to one or more periods of acoustic wave energy followed by incubation in the absence of acoustic stimulation.

29. The method of claim 27, wherein the cell, or a population thereof, is exposed to acoustic wave energy once an hour for at least 2 hours.

30. The method of claim 27, The method of claim 27, wherein the cell or a population thereof, is exposed to acoustic wave energy once an hour for a period between about 30 seconds to about 60 minutes, for a period between about 30 seconds to about 30 minutes, for a period of about 5 minutes, or for a period of about 10 minutes.

31. The method of any one of claims 1 to 30, wherein the cell is a mammalian cell.

32. The method of any one of claims 1 to 31, wherein the cell is a human cell.

33. A method of increasing an immune response in a subject, the method comprising performing the method of any one of claims 2 or 4 to 32, and administering the cell, or a population thereof, to the subject.

34. The method of claim 33, wherein the subject has a cancer, an infection, or an inflammatory disease.

35. The method of claim 34, wherein the subject has cancer and the cell or population thereof expressed a CAR.

36. A method of in vitro fertilisation, the method comprising performing the method of any one of claims 3 or 9 to 32, and implanting the fertilised ovum into the reproductive tract of a female subject.

37. A method of artificial insemination, the method comprising exposing sperm to acoustic wave energy, and implanting the sperm into the reproductive tract of a female subject.

38. A method of differentiating a monocyte into a macrophage, the method comprising exposing the monocyte to acoustic wave energy.

39. The method of claim 38, wherein differentiation is characterised by one or more or all of phagocytotic ability, adherence ability, and/or surface marker expression.

40. The method of claim 30, wherein the subject has cancer and the cell or population thereof is a monocyte or a population of monocytes that have been differentiated into a macrophage or a population of macrophages.

41. A cell produced using the method of any one of claims 1 to 40.


SEQUENCE LISTING (6 sequences)

SEQ ID NO: 1 - DNA, 22 bp, synthetic construct
  source 1..22: mol_type = other DNA
catgttccaatatgattccacc

SEQ ID NO: 2 - DNA, 21 bp, synthetic construct
  source 1..21: mol_type = other DNA
gatgggatttccattgatgac

SEQ ID NO: 3 - DNA, 20 bp, synthetic construct
  source 1..20: mol_type = other DNA
gcagtcctgctctttgaacc

SEQ ID NO: 4 - DNA, 20 bp, synthetic construct
  source 1..20: mol_type = other DNA
ctgcttgtccacacgaagaa

SEQ ID NO: 5 - DNA, 21 bp, synthetic construct
  source 1..21: mol_type = other DNA
gaaccagatgcacattgatga

SEQ ID NO: 6 - DNA, 21 bp, synthetic construct
  source 1..21: mol_type = other DNA
cgactcacgcttgacttgatg